(19) United States
(12) Patent Application Publication
(10) Pub. No.: US 2022/0409608 A1
(43) Pub. Date: Dec. 29, 2022

(54) DIMERIC NAPHTHALIMIDE FORMULATIONS

(71) Applicant: ALUCENT BIOMEDICAL, INC., Salt Lake City, UT (US)

(72) Inventor: Kevin S. Warner, Salt Lake City, UT (US)

(73) Assignee: ALUCENT BIOMEDICAL, INC., Salt Lake City, UT (US)

(21) Appl. No.: 17/772,456

(22) PCT Filed: Oct. 28, 2020
(86) PCT No.: PCT/US2020/057659

§ 371 Date: Apr. 27, 2022

Related U.S. Application Data
(60) Provisional application No. 62/927,430, filed on Oct. 29, 2019.

(51) Int. Cl. A61K 31/473; A61K 31/337

(52) U.S. Cl. CPC A61K 31/473; A61K 31/337

(57) ABSTRACT

The present disclosure provides novel formulations for dimeric naphthalimides and stability studies of the same. In some embodiments, the dimeric naphthalimide is a diacetate salt of Compound of Formula (I).

The present disclosure provides novel stable formulations for dimeric naphthalimides.

Certain dimeric naphthalimide compounds have been previously disclosed. See, e.g., U.S. Pat. No. 6,410,505 B2. For example, a dimeric naphthalimide compound, 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione), also known as 10-8-10 dimer, 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbix(oxy-2,1-ethanediyl)]bis[6-({2-[2-(2-aminoethoxy)ethoxy]ethyl}amino)-1H-benzo[de]isoquinoline-1,3(2H)-dione]; and 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]ethyl]amino]-(9Cl), and herein referred to as Compound of Formula (I), has been disclosed. Id. Solid state forms of Compound of Formula (I), as well as methods for making the same, have been described in U.S. Provisional Patent Application No. 62/785,477.

The present disclosure provides novel formulations comprising at least one active agent chosen from a Compound of Formula (I):

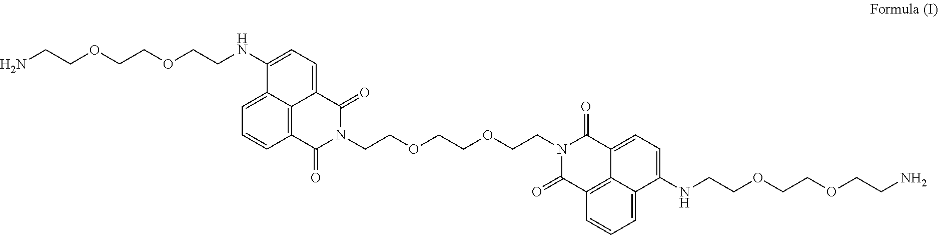 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

and pharmaceutically acceptable salts thereof.

It was surprisingly and unpredictably discovered that the Compound of Formula (I) and pharmaceutically acceptable salts thereof degrade appreciably in compositions having a pH of less than 5 or greater than 7. Moreover, it was surprisingly and unpredictably discovered that, in a composition comprising the Compound of Formula (I) and/or pharmaceutically acceptable salts thereof at a pH ranging from 5 to 7, the increase in impurity concentration was minimal.

In some embodiments, provided herein is a composition comprising at least one active agent, at least one tonicity agent, at least one buffer, and at least one vehicle. In some embodiments, the at least one active agent is chosen from a Compound of Formula (I) and pharmaceutically acceptable salts thereof.

### BRIEF DESCRIPTION OF DRAWINGS

FIG. 1 shows the total amount of impurities in a composition described herein at 40° C. at various pH values as a function of time.

FIG. 2 is another view of FIG. 1.

FIG. 3 shows the total amount of impurities in a composition described herein at 40° C. at 0 and 4 week time points as a function of composition pH.

FIG. 4 shows the total amount of impurities in a composition described herein at 40° C. after storage for 8 weeks at various pH values.

FIG. 5 shows HPLC chromatograms of a mixture of Compound of Formula (I) and radiographic contrast agent Isovue at initial and 24-hour timepoints. The chromatograms show no increase in the amount of impurities of Compound of Formula (I) over the time studied.

FIG. 6 shows HPLC chromatograms of a mixture of Compound of Formula (I) and radiographic contrast agent Omnipaque at initial and 24-hour timepoints. The chromatograms show no increase in the amount of impurities of Compound of Formula (I) over the time studied.

FIG. 7 shows HPLC chromatograms of Compound of Formula (I), and a mixture of Compound of Formula (I) and paclitaxel at 1- and 7-hour time points after preparing the mixture. The chromatograms show no increase in the amount of impurities of Compound of Formula (I) over the time studied.

FIG. 8 shows HPLC chromatograms of paclitaxel, and a mixture of Compound of Formula (I) and paclitaxel at 1- and 7-hour time points after preparing the mixture. The chromatograms show no increase in the amount of impurities of paclitaxel over the time studied.

As used herein, the following definitions shall apply unless otherwise indicated.

As used herein, the singular terms “a,” “an,” and “the” include the plural reference unless the context clearly indicates otherwise.

The phrase “and/or,” as used herein, means “either or both” of the elements so conjoined, i.e., elements that are conjunctively present in some cases and disjunctively present in other cases. Thus, as a non-limiting example, “A and/or B”, when used in conjunction with open-ended language such as “comprising” can refer, in some embodiments, to A only (optionally including elements other than B); in other embodiments, to B only (optionally including elements other than A); in yet other embodiments, to both A and B (optionally including other elements); etc.

As used herein, “Compound of Formula (I)” includes one or more of the conformational forms of the compound. Unless stated otherwise, compounds depicted herein coexisting with tautomeric forms are within the scope of the disclosure. Additionally, unless stated otherwise, structures depicted herein are also meant to include compounds that differ only in the presence of one or more isotopically enriched atoms. For example, compounds having the depicted structures except for the replacement of hydrogen by deuterium or tritium, or the replacement of a carbon atom by ¹³C- or ¹⁴C-enriched carbon atom are within the scope of this disclosure.

The Compound of Formula (I) may be described by the structure:

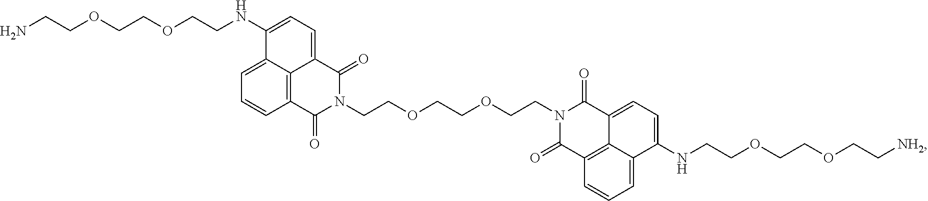 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

by the chemical names 2,2′-((ethane-1,2-diylbis(oxy))bis(ethane-2,1-diyl))bis(6-((2-(2-(2-aminoethoxy)ethoxy)ethyl)amino)-1H-benzo[de]isoquinoline-1,3(2H)-dione); 6-[2-[2-(2-aminoethoxy)ethoxy]ethylamino]-2-[2-[2-[2-[6-[2-[2-(2-aminoethoxy)ethoxy]ethylamino]-1,3-dioxobenzo[de]isoquinolin-2-yl]ethoxy]ethoxy]ethyl]benzo[de]isoquinoline-1,3-dione; 2,2′-[1,2-ethanediylbix(oxy-2,1-ethanediyl)]bis[6-({2-[2-(2-aminoethoxy)ethoxy]ethyl}-amino)-1H-benzo[de]isoquinoline-1,3(2H)-dione]; or 1H-benz[de]isoquinoline-1,3(2H)-dione, 2,2′-[1,2-ethanediylbis(oxy-2,1-ethanediyl)]bis[6-[[2-[2-(2-aminoethoxy)ethoxy]-ethyl]amino]-(9Cl), or by the Chemical Abstract Services (CAS) Registry No. 438200-66-9.

“Pharmaceutically acceptable” as used herein to modify a carrier, vehicle, and/or excipient, refers to a nontoxic carrier, vehicle, and/or excipient, respectively, that does not destroy the pharmacological activity of the compound with which it is formulated.

As used herein, the term “preparation of the composition” means the point in time at which all of the formulation components have been combined.

As used herein, the term “use of the composition” means administration to a subject, which includes humans and animals, or a biological system in need thereof.

As used herein, the terms “degrade”, “degrades”, and “degradation” may be used interchangeably to refer to the chemical conversion of a chemical entity, e.g., the at least one active agent, into another chemical entity. In some embodiments, degradation of one chemical entity may result in the appearance or increase in the amount of one or more impurities, which may have undesired effect(s) on a subject who has been administered the one or more impurities.

The degradation of a chemical entity, e.g., the at least one active agent, may be assessed using methods commonly employed in the art to detect and/or distinguish chemical compounds, e.g., liquid chromatography (“LC”) and/or mass spectrometry.

When a chemical entity, e.g., the at least one active agent, is described herein as “not degrading” after a certain time period, that means that there is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 98.5%, at least 98.75%, at least 99%, at least 99.1%, at least 99.2%, at least 99.25%, at least 99.3%, at least 99.35%, at least 99.4%, at least 99.45%, at least 99.5%, at least 99.55%, at least 99.6%, at least 99.65%, at least 99.7%, at least 99.75%, at least 99.8%, at least 99.85%, at least 99.9%, or at least 99.95% of the chemical entity remaining in the composition at the specified time point. In some embodiments, degradation is determined by LC.

As used therein, the term “impurity” refers to an unwanted chemical entity in a composition. The genesis of the impurity may be from degradation of a component of a composition described herein, e.g., degradation of the at least one active agent, or the impurity may have been introduced during the preparation of the composition. The presence of one or more impurities may be assessed using methods commonly employed in the art to detect and/or distinguish chemical compounds, e.g., liquid chromatography (“LC”) and/or mass spectrometry.

In some embodiments, the compositions described herein contain a total amount of impurities of less than 15%, less than 14%, less than 13%, less than 12%, less than 11%, less than 10%, less than 9%, less than 8%, less than 7%, less than 6%, less than 5%, less than 4%, less than 3%, less than 2.5%, less than 2%, less than 1.5%, less than 1.25%, less than 1%, less than 0.95%, less than 0.9%, less than 0.85%, less than 0.8%, less than 0.75%, less than 0.7%, less than 0.65%, less than 0.6%, less than 0.55%, less than 0.5%, less than 0.45%, less than 0.4%, less than 0.35%, less than 0.3%, less than 0.25%, less than 0.2%, less than 0.15%, less than 0.1%, or less than 0.05% by weight of the composition. In some embodiments, the amount of impurities present in the composition is determined by LC.

As used herein, the term “LC” means liquid chromatography and includes “HPLC” and “UPLC”, which refer to high performance liquid chromatography and ultra performance liquid chromatography, respectively.

In some embodiments, the weight percent of the Compound of Formula (I) in a composition can be determined based on LC analysis according to the formula:

$$Percent Assay = \frac{{A}_{Samp}}{{A}_{std}} \times \frac{{C}_{std} \times 10}{{C}_{calc}} \times 100$$

wherein,
A_{Samp}=Peak area response of Compound of Formula (I) in sample injection
A_{Std}=Mean peak area response of Compound of Formula (I) in all working standard injections
C_{Std}=Concentration of standard, in mg/mL
C_{calc}=Calculated concentration of Compound of Formula (I) in composition, in mg/mL

In some embodiments, the weight percent of the impurities in a composition can be determined based on LC analysis according to the formula:

$$Percent Impurities ( \% - area ) = \frac{{A}_{S ampI}}{A} \times \times 100$$

wherein,
A_{SampI}=Peak area response of impurity in sample injection
A_{I}=Mean peak area response of Compound of Formula (I) in impurity level standard injection

As used herein, the term “radiographic contrast agent” refers to a pharmaceutically acceptable agent used to increase the contrast of structures or fluids within a subject during a medical imaging procedure. Non-limiting exemplary radiographic contrast agents include Isovue, Omnipaque, and Visipaque.

As used herein, the term “pharmaceutically acceptable agent” refers to a chemical entity that does not adversely impact the pharmacological activity of the compound with which it is formulated.

As used herein, the term “anti-proliferative agent” refers to a pharmaceutically acceptable agent capable of disrupting a cell's division cycle. Exemplary anti-proliferative agents include, but are not limited to, paclitaxel, paclitaxel derivatives (e.g., docetaxel and cabazitaxel), rapamycin, rapamycin derivatives (e.g., everolimus, ridaforolimus, tacrolimus, umirolimus, and zotarolimus), and pharmaceutically acceptable salts thereof.

At Least One Active Agent

Provided herein is a composition comprising at least one active agent. In some embodiments, the at least one active agent is chosen from dimeric naphthalimides and pharmaceutically acceptable salts thereof, e.g., the dimeric naphthalimides disclosed in U.S. Pat. No. 6,410,505 B2. In some embodiments, the at least one active agent is a Compound of Formula (I). In some embodiments, the at least one active agent is chosen from a Compound of Formula (I) and pharmaceutically acceptable salts thereof.

In some embodiments, the at least one active agent is present in an amount ranging from 0.01% to 5% by weight of the composition. In some embodiments, the at least one active agent is present in an amount ranging from 0.01% to 4% by weight of the composition. In some embodiments, the at least one active agent is present in an amount ranging from 0.01% to 2.5% by weight of the composition. In some embodiments, the at least one active agent is present in an amount of 0.01%, 0.03%, 0.05%, 0.07%, 0.09%, 0.11%, 0.13%, 0.15%, 0.17%, 0.19%, 0.21%, 0.23%, 0.25%, 0.27%, 0.29%, 0.31%, 0.33%, 0.35%, 0.37%, 0.39%, 0.41%, 0.43%, 0.45%, 0.47%, 0.49%, 0.51%, 0.53%, 0.55%, 0.57%, 0.59%, 0.61%, 0.63%, 0.65%, 0.67%, 0.69%, 0.71%, 0.73%, 0.75%, 0.77%, 0.79%, 0.81%, 0.83%, 0.85%, 0.87%, 0.89%, 0.91%, 0.93%, 0.95%, 0.97%, 0.99%, 1.01%, 1.03%, 1.05%, 1.07%, 1.09%, 1.11%, 1.13%, 1.15%, 1.17%, 1.19%, 1.21%, 1.23%, 1.25%, 1.27%, 1.29%, 1.31%, 1.33%, 1.35%, 1.37%, 1.39%, 1.41%, 1.43%, 1.45%, 1.47%, 1.49%, 1.51%, 1.53%, 1.55%, 1.57%, 1.59%, 1.61%, 1.63%, 1.65%, 1.67%, 1.69%, 1.71%, 1.73%, 1.75%, 1.77%, 1.79%, 1.81%, 1.83%, 1.85%, 1.87%, 1.89%, 1.91%, 1.93%, 1.95%, 1.97%, 1.99%, 2.01%, 2.03%, 2.05%, 2.07%, 2.09%, 2.11%, 2.13%, 2.15%, 2.17%, 2.19%, 2.21%, 2.23%, 2.25%, 2.27%, 2.29%, 2.31%, 2.33%, 2.35%, 2.37%, 2.39%, 2.41%, 2.43%, 2.45%, 2.47%, or 2.49% by weight of the composition. In some embodiments, the at least one active agent is present in an amount of 0.1%, 0.2%, 0.3%, 0.4%, 0.5%, 0.6%, 0.7%, 0.8%, 0.9%, 1%, 1.1%, or 1.2% by weight of the composition.

In some embodiments, the at least one active agent is present in an amount ranging from 0.01% to 1% by weight of the composition. In some embodiments, the at least one active agent is present in an amount ranging from 0.3% to 0.6% by weight of the composition. In some embodiments, the at least one active agent is present in an amount ranging from 0.4% to 0.5% by weight of the composition. In some embodiments, the at least one active agent is present in an amount ranging from 0.1% to 0.5% by weight of the composition. In some embodiments, the at least one active agent is present in an amount of 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, or 0.5% by weight of the composition. In some embodiments, the at least one active agent is present in an amount ranging from 0.1% to 0.3% by weight of the composition.

In some embodiments, the at least one active agent is present in an amount of 0.01%, 0.02%, 0.03%, 0.04%, 0.05%, 0.06%, 0.07%, 0.08%, 0.09%, 0.1%, 0.12%, 0.14%, 0.16%, 0.18%, 0.2%, 0.22%, 0.24%, 0.26%, 0.28%, 0.3%, 0.32%, 0.34%, 0.36%, 0.38%, 0.4%, 0.42%, 0.44%, 0.48%, 0.5%, 0.52%, 0.54%, 0.56%, 0.58%, 0.6%, 0.62%, 0.64%, 0.66%, 0.68%, 0.7%, 0.72%, 0.74%, 0.76%, 0.78%, 0.8%, 0.82%, 0.84%, 0.86%, 0.88%, 0.9%, 0.92%, 0.94%, 0.96%, 0.98%, or 1% by weight of the composition.

In some embodiments, the at least one active agent is present in an amount of 0.08% by weight of the composition. In some embodiments, the at least one active agent is present in an amount of 0.1% by weight of the composition. In some embodiments, the at least one active agent is present in an amount of 0.12% by weight of the composition. In some embodiments, the at least one active agent is present in an amount of 0.14% by weight of the composition. In some embodiments, the at least one active agent is present in an amount of 0.16% by weight of the composition. In some embodiments, the at least one active agent is present in an amount of 0.18% by weight of the composition. In some embodiments, the at least one active agent is present in an amount of 0.2% by weight of the composition. In some embodiments, the at least one active agent is present in an amount of 0.22% by weight of the composition. In some embodiments, the at least one active agent is present in an amount of 0.24% by weight of the composition. In some embodiments, the at least one active agent is present in an amount of 0.26% by weight of the composition. In some embodiments, the at least one active agent is present in an amount of 0.36% by weight of the composition. In some embodiments, the at least one active agent is present in an amount of 0.38% by weight of the composition. In some embodiments, the at least one active agent is present in an amount of 0.4% by weight of the composition. In some embodiments, the at least one active agent is present in an amount of 0.42% by weight of the composition. In some embodiments, the at least one active agent is present in an amount of 0.44% by weight of the composition. In some embodiments, the at least one active agent is present in an amount of 0.46% by weight of the composition. In some embodiments, the at least one active agent is present in an amount of 0.48% by weight of the composition. In some embodiments, the at least one active agent is present in an amount of 0.5% by weight of the composition.

At Least One Tonicity Agent

In some embodiments, the composition further comprises at least one tonicity agent. Without being bound by any theory, in some embodiments, at least one tonicity agent may be added to modulate the solute concentration of a composition.

In some embodiments, the at least one tonicity agent is chosen from dextrose, sorbitol, lactose, mannitol, sodium chloride, potassium chloride, and glycerol. In some embodiments, the at least one tonicity agent is chosen from sodium chloride and potassium chloride. In some embodiments, the at least one tonicity agent is sodium chloride and potassium chloride.

In some embodiments, the at least one tonicity agent is dextrose. In some embodiments, the at least one tonicity agent is sorbitol. In some embodiments, the at least one tonicity agent is lactose. In some embodiments, the at least one tonicity agent is mannitol. In some embodiments, the at least one tonicity agent is sodium chloride. In some embodiments, the at least one tonicity agent is potassium chloride. In some embodiments, the at least one tonicity agent is glycerol.

In some embodiments, the at least one tonicity agent is present in an amount up to 5% by weight of the composition. In some embodiments, the at least one tonicity agent is present in an amount up to 4% by weight of the composition. In some embodiments, the at least one tonicity agent is present in an amount up to 3% by weight of the composition. In some embodiments, the at least one tonicity agent is present in an amount of up to 2.5% by weight of the composition. In some embodiments, the at least one tonicity agent is present in an amount of up to 2% by weight of the composition. In some embodiments, the at least one tonicity agent is present in an amount of up to 1.5% by weight of the composition. In some embodiments, the at least one tonicity agent is present in an amount of up to 1% by weight of the composition. In some embodiments, the at least one tonicity agent is present in an amount of up to 0.5% by weight of the composition.

In some embodiments, the at least one tonicity agent is present in an amount ranging from 0.25% to 3% by weight of the composition. In some embodiments, the at least one tonicity agent is present in an amount ranging from 0.25% to 2.5% by weight of the composition. In some embodiments, the at least one tonicity agent is present in an amount ranging from 0.5% to 2% by weight of the composition. In some embodiments, the at least one tonicity agent is present in an amount ranging from 0.5% to 1.5% by weight of the composition.

In some embodiments, the at least one tonicity agent is present in an amount of 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, or 0.9% by weight of the composition. In some embodiments, the at least one tonicity agent is present in an amount of 0.72%, 0.74%, 0.76%, 0.78%, 0.8%, 0.82%, or 0.84% by weight of the composition. In some embodiments, the at least one tonicity agent is present in an amount of 0.72% by weight of the composition. In some embodiments, the at least one tonicity agent is present in an amount of 0.74% by weight of the composition. In some embodiments, the at least one tonicity agent is present in an amount of 0.76% by weight of the composition. In some embodiments, the at least one tonicity agent is present in an amount of 0.78% by weight of the composition. In some embodiments, the at least one tonicity agent is present in an amount of 0.8% by weight of the composition. In some embodiments, the at least one tonicity agent is present in an amount of 0.82% by weight of the composition. In some embodiments, the at least one tonicity agent is present in an amount of 0.84% by weight of the composition.

At Least One Buffer

In some embodiments, the composition further comprises at least one buffer. Without being bound by any theory, in some embodiments, at least one buffer may be added to maintain a desired pH or pH range.

In some embodiments, the at least one buffer is chosen from a potassium salt, a sodium salt, and maleic acid. In some embodiments, the at least one buffer is a sodium salt. In some embodiments, the at least one buffer is a potassium salt. In some embodiments, the at least one buffer is maleic acid. In some embodiments, the at least one buffer comprises a potassium salt and a sodium salt.

In some embodiments, the potassium salt is chosen from potassium phosphate, potassium citrate, potassium acetate, potassium lactate, and potassium tartrate. In some embodiments, the sodium salt is chosen from sodium phosphate, sodium citrate, sodium acetate, sodium lactate, and sodium tartrate. In some embodiments, the at least one buffer is chosen from potassium phosphate and sodium phosphate. In some embodiments, the at least one buffer comprises potassium phosphate and sodium phosphate.

In some embodiments, the potassium phosphate is chosen from potassium phosphate monobasic, potassium phosphate dibasic, and potassium phosphate tribasic. In some embodiments, the potassium phosphate is chosen from potassium phosphate monobasic and potassium phosphate dibasic. In some embodiments, the potassium phosphate is potassium phosphate monobasic. In some embodiments, the potassium phosphate is potassium phosphate dibasic. In some embodiments, the potassium phosphate is potassium phosphate tribasic.

In some embodiments, the sodium phosphate is chosen from sodium phosphate monobasic, sodium phosphate dibasic, and sodium phosphate tribasic.

In some embodiments, the sodium phosphate is chosen from sodium phosphate monobasic and sodium phosphate dibasic. In some embodiments, the sodium phosphate is sodium phosphate monobasic. In some embodiments, the sodium phosphate is sodium phosphate dibasic. In some embodiments, the sodium phosphate is sodium phosphate tribasic.

In some embodiments, the at least one buffer is chosen from sodium phosphate dibasic and potassium phosphate monobasic. In some embodiments, the at least one buffer is sodium phosphate dibasic and potassium phosphate monobasic.

In some embodiments, the at least one buffer is anhydrous.

In some embodiments, the at least one buffer is present in an amount of up to 2.5% by weight of the composition. In some embodiments, the at least one buffer is present in an amount of up to 2% by weight of the composition. In some embodiments, the at least one buffer is present in an amount of up to 1.5% by weight of the composition. In some embodiments, the at least one buffer is present in an amount of up to 1% by weight of the composition. In some embodiments, the at least one buffer is present in an amount of up to 0.5% by weight of the composition.

In some embodiments, the at least one buffer is present in an amount ranging from 0.05% to 0.4% by weight of the composition. In some embodiments, the at least one buffer is present in an amount of 0.06%, 0.08%, 0.1%, 0.12%, 0.14%, 0.16%, 0.18% 0.2%, 0.22%, 0.24%, 0.26%, 0.28%, or 0.3% by weight of the composition. In some embodiments, the at least one buffer is present in an amount ranging from 0.1% to 0.2% by weight of the composition. In some embodiments, the at least one buffer is present in an amount ranging from 0.08% to 0.16% by weight of the composition. In some embodiments, the at least one buffer is present in an amount of 0.1% by weight of the composition. In some embodiments, the at least one buffer is present in an amount of 0.11% by weight of the composition. In some embodiments, the at least one buffer is present in an amount of 0.12% by weight of the composition. In some embodiments, the at least one buffer is present in an amount of 0.13% by weight of the composition. In some embodiments, the at least one buffer is present in an amount of 0.14% by weight of the composition. In some embodiments, the at least one buffer is present in an amount of 0.15% by weight of the composition. In some embodiments, the at least one buffer is present in an amount of 0.16% by weight of the composition. In some embodiments, the at least one buffer is present in an amount of 0.17% by weight of the composition. In some embodiments, the at least one buffer is present in an amount of 0.18% by weight of the composition. In some embodiments, the at least one buffer is present in an amount of 0.19% by weight of the composition. In some embodiments, the at least one buffer is present in an amount of 0.2% by weight of the composition.

In some embodiments, the at least one buffer comprises sodium phosphate in an amount ranging from 0.01% to 0.03% by weight of the composition and potassium phosphate in an amount ranging from 0.1% to 0.14% by weight of the composition.

At Least One Vehicle

In some embodiments, provided herein is a composition comprising at least one vehicle. In some embodiments, the at least one vehicle is chosen from water, ethanol, isopropanol, polyethylene glycols, and propylene glycols.

In some embodiments, the at least one vehicle is water. In some embodiments, the at least one vehicle is ethanol. In some embodiments, the at least one vehicle is isopropanol. In some embodiments, the at least one vehicle is chosen from polyethylene glycols. In some embodiments, the at least one vehicle is chosen from propylene glycols.

In some embodiments, the polyethylene glycols are chosen from polyethylene glycol 300, polyethylene glycol 400, polyethylene glycol 500, and polyethylene glycol 600.

In some embodiments, the at least one vehicle is present in an amount of qs 100% by weight of the composition.

At Least One pH Modulation Agent

In some embodiments, the composition further comprises at least one pH modulation agent. Without being bound by any theory, in some embodiments, at least one pH modulation agent may be added to achieve a desired pH of the composition.

In some embodiments, the at least one pH modulation agent is chosen from acetic acid, carbonic acid, citric acid, sodium bicarbonate, and sodium hydroxide. In some embodiments, the at least one pH modulation agent is acetic acid. In some embodiments, the at least one pH modulation agent is carbonic acid. In some embodiments, the at least one pH modulation agent is citric acid. In some embodiments, the at least one pH modulation agent is sodium bicarbonate. In some embodiments, the at least one pH modulation agent is sodium hydroxide.

In some embodiments, the at least one pH modulation agent is chosen from acetic acid and sodium hydroxide. In some embodiments, the at least one pH modulation agent is acetic acid and sodium hydroxide.

In some embodiments, the at least one pH modulation agent is present in an amount of qs to desired pH.

At Least One Viscosity Agent

In some embodiments, the composition further comprises at least one viscosity agent. Without being bound by any theory, in some embodiments, at least one viscosity agent may be added to achieve a desired viscosity or thickness of the composition.

In some embodiments, the at least one viscosity agent is chosen from gelatin, methylcellulose, hydroxypropyl cellulose, hydroxypropyl methylcellulose, hydroxyethyl cellulose, hydroxypropylmethyl cellulose, methylhydroxyethyl cellulose, methylhydroxypropyl cellulose, hydroxyethylcarboxymethyl cellulose, carboxymethyl cellulose, carboxymethylhydroxyethyl cellulose, and sodium carboxymethylcellulose. In some embodiments, the at least one viscosity agent is chosen from methylcellulose, sodium carboxymethylcellulose, and hydroxypropyl cellulose.

In some embodiments, the at least one viscosity agent is gelatin. In some embodiments, the at least one viscosity agent is methylcellulose. In some embodiments, the at least one viscosity agent is hydroxypropyl cellulose. In some embodiments, the at least one viscosity agent is hydroxypropyl methylcellulose. In some embodiments, the at least one viscosity agent is hydroxyethyl cellulose. In some embodiments, the at least one viscosity agent is hydroxypropylmethyl cellulose. In some embodiments, the at least one viscosity agent is methylhydroxyethyl cellulose. In some embodiments, the at least one viscosity agent is methylhydroxypropyl cellulose. In some embodiments, the at least one viscosity agent is hydroxyethylcarboxymethyl cellulose. In some embodiments, the at least one viscosity agent is carboxymethyl cellulose. In some embodiments, the at least one viscosity agent is carboxymethylhydroxyethyl cellulose. In some embodiments, the at least one viscosity agent is sodium carboxymethylcellulose.

In some embodiments, the at least one viscosity agent is present in an amount of 2.5% or less by weight of the composition. In some embodiments, the at least one viscosity agent is present in an amount of 2% or less by weight of the composition. In some embodiments, the at least one viscosity agent is present in an amount of 1.5% or less by weight of the composition. In some embodiments, the at least one viscosity agent is present in an amount of 1% or less by weight of the composition. In some embodiments, the at least one viscosity agent is present in an amount of 0.5% or less by weight of the composition.

In some embodiments, the at least one viscosity agent is present in an amount ranging from 0.05% to 1% by weight of the composition. In some embodiments, the at least one viscosity agent is present in an amount ranging from 0.1% to 0.5% by weight of the composition. In some embodiments, the at least one viscosity agent is present in an amount ranging from 0.1% to 0.3% by weight of the composition. In some embodiments, the at least one viscosity agent is present in an amount of 0.15%, 0.16%, 0.17%, 0.18%, 0.19%, 0.2%, 0.21%, 0.22%, 0.23%, 0.24%, or 0.25% by weight of the composition.

In some embodiments, the at least one viscosity agent is present in an amount of 0.15% by weight of the composition. In some embodiments, the at least one viscosity agent is present in an amount of 0.16% by weight of the composition. In some embodiments, the at least one viscosity agent is present in an amount of 0.17% by weight of the composition. In some embodiments, the at least one viscosity agent is present in an amount of 0.18% by weight of the composition. In some embodiments, the at least one viscosity agent is present in an amount of 0.19% by weight of the composition. In some embodiments, the at least one viscosity agent is present in an amount of 0.2% by weight of the composition. In some embodiments, the at least one viscosity agent is present in an amount of 0.21% by weight of the composition. In some embodiments, the at least one viscosity agent is present in an amount of 0.22% by weight of the composition. In some embodiments, the at least one viscosity agent is present in an amount of 0.23% by weight of the composition. In some embodiments, the at least one viscosity agent is present in an amount of 0.24% by weight of the composition. In some embodiments, the at least one viscosity agent is present in an amount of 0.25% by weight of the composition.

At Least One Penetration Enhancer

In some embodiments, the composition further comprises at least one penetration enhancer. Without being bound by any theory, in some embodiments, at least one penetration enhancer may be added to increase the amount of the at least one active agent delivered to the desired location.

In some embodiments, the at least one penetration enhancer is chosen from benzyl alcohol, diethylene glycol monoethyl ether, caprylic acid, and sodium oleate. In some embodiments, the at least one penetration enhancer is benzyl alcohol. In some embodiments, the at least one penetration enhancer is diethylene glycol monoethyl ether. In some embodiments, the at least one penetration enhancer is caprylic acid. In some embodiments, the at least one penetration enhancer is sodium oleate.

In some embodiments, the at least one penetration enhancer is present in an amount ranging from 0.01% to 1% by weight of the composition. In some embodiments, the at least one penetration enhancer is present in an amount ranging from 0.01% to 0.5% by weight of the composition. In some embodiments, the at least one penetration enhancer is present in an amount ranging from 0.05% to 0.25% by weight of the composition. In some embodiments, the at least one penetration enhancer is present in an amount of 0.05%, 0.1%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, or 1% by weight of the composition.

At Least One Stabilizing Agent

In some embodiments, the composition further comprises at least one stabilizing agent. Without being bound by any theory, in some embodiments, at least one stabilizing agent may be added to retard or completely prevent degradation of the at least one active agent and/or the stabilizing agent may retard or completely prevent the appearance of impurities in the composition.

In some embodiments, the at least one stabilizing agent is chosen from ascorbic acid, butylated hydroxytoluene, citric acid, benzoic acid, and sodium metabisulfite. In some embodiments, the at least one stabilizing agent is ascorbic acid. In some embodiments, the at least one stabilizing agent is butylated hydroxytoluene. In some embodiments, the at least one stabilizing agent is citric acid. In some embodiments, the at least one stabilizing agent is benzoic acid. In some embodiments, the at least one stabilizing agent is sodium metabisulfite.

In some embodiments, the at least one stabilizing agent is present in an amount ranging from 0.005% to 1% by weight of the composition. In some embodiments, the at least one stabilizing agent is present in an amount ranging from 0.005% to 0.25%, 0.5%, 0.75%, or 1% by weight of the composition.

In some embodiments, the at least one stabilizing agent is present in an amount ranging from 0.01% to 1% by weight of the composition. In some embodiments, the at least one stabilizing agent is present in an amount ranging from 0.01% to 0.25%, 0.5%, 0.75%, or 1% by weight of the composition

In some embodiments, the at least one stabilizing agent is present in an amount ranging from 0.1% to 1% by weight of the composition. In some embodiments, the at least one stabilizing agent is present in an amount ranging from 0.1% to 0.25%, 0.5%, 0.75%, or 1% by weight of the composition.

In some embodiments, the at least one stabilizing agent is present in an amount of 0.005%, 0.01%, 0.025%, 0.05%, 0.1%, 0.15%, 0.2%, 0.25%, 0.3%, 0.4%, 0.5%, 0.75%, or 1% by weight of the composition. In some embodiments, the at least one stabilizing agent is present in an amount of 0.005% by weight of the composition. In some embodiments, the at least one stabilizing agent is present in an amount of 0.01% by weight of the composition. In some embodiments, the at least one stabilizing agent is present in an amount of 0.025% by weight of the composition. In some embodiments, the at least one stabilizing agent is present in an amount of 0.05% by weight of the composition. In some embodiments, the at least one stabilizing agent is present in an amount of 0.1% by weight of the composition. In some embodiments, the at least one stabilizing agent is present in an amount of 0.15% by weight of the composition. In some embodiments, the at least one stabilizing agent is present in an amount of 0.2% by weight of the composition. In some embodiments, the at least one stabilizing agent is present in an amount of 0.25% by weight of the composition. In some embodiments, the at least one stabilizing agent is present in an amount of 0.3% by weight of the composition. In some embodiments, the at least one stabilizing agent is present in an amount of 0.4% by weight of the composition. In some embodiments, the at least one stabilizing agent is present in an amount of 0.5% by weight of the composition. In some embodiments, the at least one stabilizing agent is present in an amount of 0.75% by weight of the composition. In some embodiments, the at least one stabilizing agent is present in an amount of 1% by weight of the composition.

At Least One Solubilizing Agent

In some embodiments, the composition further comprises at least one solubilizing agent. Without being bound by any theory, in some embodiments, at least one solubilizing agent may be added to increase the solubility of the at least one active agent in a vehicle, e.g., water, by forming, e.g., an emulsion.

In some embodiments, the at least one solubilizing agent is chosen from tocopherols, fixed oils, soybean oil, PEG-15 hydroxystearates, polysorbate 20, polysorbate 80, 2-hydroxypropyl-β-cyclodextrin, and γ-cyclodextrin. In some embodiments, the solubilizing agent is chosen from tocopherols. In some embodiments, the solubilizing agent is chosen from fixed oils. In some embodiments, the solubilizing agent is chosen from PEG-15 hydroxystearates. In some embodiments, the solubilizing agent is polysorbate 20. In some embodiments, the solubilizing agent is polysorbate 80. In some embodiments, the solubilizing agent is 2-hydroxypropyl-β-cyclodextrin. In some embodiments, the solubilizing agent is γ-cyclodextrin.

In some embodiments, fixed oils are chosen from corn oil, cottonseed oil, peanut oil, and sesame oil.

In some embodiments, the at least one solubilizing agent is present in an amount ranging from 0.005% to 10% by weight of the composition. In some embodiments, the at least one solubilizing agent is present in an amount ranging from 0.005% to 0.1%, 0.25%, 0.5%, 1%, 1.5%, 2.5%, 5%, 7.5%, or 10% by weight of the composition. In some embodiments, the at least one solubilizing agent is present in an amount ranging from 0.1% to 10% by weight of the composition. In some embodiments, the at least one solubilizing agent is present in an amount ranging from 0.1% to 0.25%, 0.5%, 1%, 1.5%, 2.5%, 5%, 7.5%, or 10% by weight of the composition.

In some embodiments, the at least one solubilizing agent is present in an amount ranging from 1% to 10% by weight of the composition. In some embodiments, the at least one solubilizing agent is present in an amount ranging from 1% to 1.5%, 2.5%, 5%, 7.5%, or 10% by weight of the composition.

In some embodiments, the at least one solubilizing agent is present in an amount of 0.005%, 0.01%, 0.025%, 0.05%, 0.1%, 0.15%, 0.2%, 0.25%, 0.3%, 0.4%, 0.5%, 0.75%, 1%, 1.5%, 2%, 2.5%, 5%, 7.5%, or 10% by weight of the composition. In some embodiments, the at least one solubilizing agent is present in an amount of 0.005% by weight of the composition. In some embodiments, the at least one solubilizing agent is present in an amount of 0.01% by weight of the composition. In some embodiments, the at least one solubilizing agent is present in an amount of 0.025% by weight of the composition. In some embodiments, the at least one solubilizing agent is present in an amount of 0.05% by weight of the composition. In some embodiments, the at least one solubilizing agent is present in an amount of 0.1% by weight of the composition. In some embodiments, the at least one solubilizing agent is present in an amount of 0.15% by weight of the composition. In some embodiments, the at least one solubilizing agent is present in an amount of 0.2% by weight of the composition. In some embodiments, the at least one solubilizing agent is present in an amount of 0.25% by weight of the composition. In some embodiments, the at least one solubilizing agent is present in an amount of 0.3% by weight of the composition. In some embodiments, the at least one solubilizing agent is present in an amount of 0.4% by weight of the composition. In some embodiments, the at least one solubilizing agent is present in an amount of 0.5% by weight of the composition. In some embodiments, the at least one solubilizing agent is present in an amount of 0.75% by weight of the composition. In some embodiments, the at least one solubilizing agent is present in an amount of 1% by weight of the composition. In some embodiments, the at least one solubilizing agent is present in an amount of 1.5% by weight of the composition. In some embodiments, the at least one solubilizing agent is present in an amount of 2% by weight of the composition. In some embodiments, the at least one solubilizing agent is present in an amount of 2.5% by weight of the composition. In some embodiments, the at least one solubilizing agent is present in an amount of 5% by weight of the composition. In some embodiments, the at least one solubilizing agent is present in an amount of 7.5% by weight of the composition. In some embodiments, the at least one solubilizing agent is present in an amount of 10% by weight of the composition.

At Least One Encapsulation Agent

In some embodiments, the composition further comprises at least one encapsulation agent. Without being bound by any theory, in some embodiments, at least one encapsulation agent may be added to improve delivery, stability, and/or solubility of the at least one active agent by, e.g., encapsulating the at least active agent in a liposome or lipid particle.

In some embodiments, the at least one encapsulation agent is chosen from 1,2-dimyristoyl-sn-glycero-phosphocholine, 1,2-distearoyl-sn-glycero-3-(phosphor-rac-(1-glycerol)), 1,2-distearoyl-sn-glycero-3-phosphocholine, cholesterol, DL-dipalmitoylphosphatidylglycerol, sodium N-(carbonyl-methoxypolyethylene glycol 2000)-1,2-distearoyl-sn-glycero-3-phosphoethanolamine, sodium N-(carbonyl-methoxypolyethylene glycol 2000)-distearoyl-glycerophosphoethanolamine, DL-distearoylphosphatidylcholine, egg phospholipids, and hydrogenated soybean lecithin.

In some embodiments, the at least one encapsulation agent is 1,2-dimyristoyl-sn-glycero-phosphocholine. In some embodiments, the at least one encapsulation agent is 1,2-distearoyl-sn-glycero-3-(phosphor-rac-(1-glycerol)). In some embodiments, the at least one encapsulation agent is 1,2-distearoyl-sn-glycero-3-phosphocholine. In some embodiments, the at least one encapsulation agent is cholesterol. In some embodiments, the at least one encapsulation agent is DL-dipalmitoylphosphatidylglyce-rol. In some embodiments, the at least one encapsulation agent is sodium N-(carbonyl-methoxypolyethylene glycol 2000)-1,2-distearoyl-sn-glycero-3-phosphoethanolamine. In some embodiments, the at least one encapsulation agent is sodium N-(carbonyl-methoxypolyethylene glycol 2000)-distearoyl-glycerophosphoethanolamine. In some embodiments, the at least one encapsulation agent is DL-distearoylphosphatidylcholine. In some embodiments, the at least one encapsulation agent is an egg phospholipid. In some embodiments, the at least one encapsulation agent is hydrogenated soybean lecithin.

In some embodiments, the at least one encapsulation agent is present in an amount ranging from 0.005% to 10% by weight of the composition. In some embodiments, the at least one encapsulation agent is present in an amount ranging from 0.005% to 0.1%, 0.25%, 0.5%, 1%, 1.5%, 2.5%, 5%, 7.5%, or 10% by weight of the composition. In some embodiments, the at least one encapsulation agent is present in an amount ranging from 0.1% to 10% by weight of the composition. In some embodiments, the at least one encapsulation agent is present in an amount ranging from 0.1% to 0.25%, 0.5%, 1%, 1.5%, 2.5%, 5%, 7.5%, or 10% by weight of the composition.

In some embodiments, the at least one encapsulation agent is present in an amount ranging from 1% to 10% by weight of the composition. In some embodiments, the at least one encapsulation agent is present in an amount ranging from 1% to 1.5%, 2.5%, 5%, 7.5%, or 10% by weight of the composition.

In some embodiments, the at least one encapsulation agent is present in an amount of 0.005%, 0.01%, 0.025%, 0.05%, 0.1%, 0.15%, 0.2%, 0.25%, 0.3%, 0.4%, 0.5%, 0.75%, 1%, 1.5%, 2%, 2.5%, 5%, 7.5%, or 10% by weight of the composition. In some embodiments, the at least one encapsulation agent is present in an amount of 0.005% by weight of the composition. In some embodiments, the at least one encapsulation agent is present in an amount of 0.01% by weight of the composition. In some embodiments, the at least one encapsulation agent is present in an amount of 0.025% by weight of the composition. In some embodiments, the at least one encapsulation agent is present in an amount of 0.05% by weight of the composition. In some embodiments, the at least one encapsulation agent is present in an amount of 0.1% by weight of the composition. In some embodiments, the at least one encapsulation agent is present in an amount of 0.15% by weight of the composition. In some embodiments, the at least one encapsulation agent is present in an amount of 0.2% by weight of the composition. In some embodiments, the at least one encapsulation agent is present in an amount of 0.25% by weight of the composition. In some embodiments, the at least one encapsulation agent is present in an amount of 0.3% by weight of the composition. In some embodiments, the at least one encapsulation agent is present in an amount of 0.4% by weight of the composition. In some embodiments, the at least one encapsulation agent is present in an amount of 0.5% by weight of the composition. In some embodiments, the at least one encapsulation agent is present in an amount of 0.75% by weight of the composition. In some embodiments, the at least one encapsulation agent is present in an amount of 1% by weight of the composition. In some embodiments, the at least one encapsulation agent is present in an amount of 1.5% by weight of the composition. In some embodiments, the at least one encapsulation agent is present in an amount of 2% by weight of the composition. In some embodiments, the at least one encapsulation agent is present in an amount of 2.5% by weight of the composition. In some embodiments, the at least one encapsulation agent is present in an amount of 5% by weight of the composition. In some embodiments, the at least one encapsulation agent is present in an amount of 7.5% by weight of the composition. In some embodiments, the at least one encapsulation agent is present in an amount of 10% by weight of the composition.

At Least One Imaging Agent

In some embodiments, the composition further comprises at least one imaging agent. In some embodiments, the at least one imaging agent is a radiographic contrast agent. In some embodiments, when at least one imaging agent is present in the composition, the composition further comprising at least one imaging agent is formed by combining a composition comprising at least one imaging agent and a composition comprising at least one active agent. In some embodiments, the composition comprising at least one active agent is a composition described herein.

In some embodiments, when at least one imaging agent is present in the composition, the composition further comprising at least one imaging agent is formed by combining a composition comprising at least one imaging agent and a composition comprising at least one active agent prior to use of the composition comprising at least one active agent and further comprising at least one imaging agent. In some embodiments, when at least one imaging agent is present in the composition, the composition further comprising at least one imaging agent is formed by combining a composition comprising at least one imaging agent and a composition comprising at least one active agent 15 seconds to 24 hours prior to use of the composition comprising at least one active agent and further comprising at least one imaging agent. In some embodiments, when at least one imaging agent is present in the composition, the composition further comprising at least one imaging agent is formed by combining a composition comprising at least one imaging agent and a composition comprising at least one active agent 15 seconds to 6 hours prior to use of the composition comprising at least one active agent and further comprising at least one active agent. In some embodiments, when at least one imaging agent is present in the composition, the composition further comprising at least one imaging agent is formed by combining a composition comprising at least one imaging agent and a composition comprising at least one active agent 15 seconds to 1 hour prior to use of the composition comprising at least one active agent and further comprising at least one active agent. In some embodiments, when at least one imaging agent is present in the composition, the composition further comprising at least one imaging agent is formed by combining a composition comprising at least one imaging agent and a composition comprising at least one active agent less than 2 hours prior to use of the composition comprising at least one active agent and further comprising at least one imaging agent.

In some embodiments, when at least one imaging agent is present in the composition, the ratio of the volume of a composition comprising at least one imaging agent to the volume of a composition comprising at least one active agent ranges from 0.5:1 to 2.5:1 prior to combination. In some embodiments, when at least one imaging agent is present in the composition, the ratio of the volume of a composition comprising at least one imaging agent to the volume of a composition comprising at least one active agent ranges from 0.5:1 to 1.5:1 prior to combination. In some embodiments, when at least one imaging agent is present in the composition, the ratio of the volume of a composition comprising at least one imaging agent to the volume of a composition comprising at least one active agent ranges from 0.75:1 to 1.25:1 prior to combination.

In some embodiments, when at least one imaging agent is present in the composition, the ratio of the volume of a composition comprising at least one imaging agent to the volume of a composition comprising at least one active agent is 0.5:1 prior to combination. In some embodiments, when at least one imaging agent is present in the composition, the ratio of the volume of a composition comprising at least one imaging agent to the volume of a composition comprising at least one active agent is 0.75:1 prior to combination. In some embodiments, when at least one imaging agent is present in the composition, the ratio of the volume of a composition comprising at least one imaging agent to the volume of a composition comprising at least one active agent is 1:1 prior to combination. In some embodiments, when at least one imaging agent is present in the composition, the ratio of the volume of a composition comprising at least one imaging agent to the volume of a composition comprising at least one active agent is 1.25:1 prior to combination. In some embodiments, when at least one imaging agent is present in the composition, the ratio of the volume of a composition comprising at least one imaging agent to the volume of a composition comprising at least one active agent is 1.5:1 prior to combination. In some embodiments, when at least one imaging agent is present in the composition, the ratio of the volume of a composition comprising at least one imaging agent to the volume of a composition comprising at least one active agent is 1.75:1 prior to combination. In some embodiments, when at least one imaging agent is present in the composition, the ratio of the volume of a composition comprising at least one imaging agent to the volume of a composition comprising at least one active agent is 2:1 prior to combination. In some embodiments, when at least one imaging agent is present in the composition, the ratio of the volume of a composition comprising at least one imaging agent to the volume of a composition comprising at least one active agent is 2.25:1 prior to combination. In some embodiments, when at least one imaging agent is present in the composition, the ratio of the volume of a composition comprising at least one imaging agent to the volume of a composition comprising at least one active agent is 2.5:1 prior to combination.

In some embodiments, when at least one imaging agent is present in the composition, the molar ratio of the at least one imaging agent to the at least one active agent ranges from 300:1 to 50:1. In some embodiments, when at least one imaging agent is present in the composition, the molar ratio of the at least one imaging agent to the at least one active agent ranges from 250:1 to 75:1. In some embodiments, when at least one imaging agent is present in the composition, the molar ratio of the at least one imaging agent to the at least one active agent ranges from 200:1 to 100:1. In some embodiments, when at least one imaging agent is present in the composition, the molar ratio of the at least one imaging agent to the at least one active agent ranges from 175:1 to 125:1. In some embodiments, when at least one imaging agent is present in the composition, the molar ratio of the at least one imaging agent to the at least one active agent is 158:1.

At Least One Anti-Proliferative Agent

In some embodiments, the composition further comprises at least one anti-proliferative agent. In some embodiments, when at least one anti-proliferative agent is present in the composition, the composition further comprising at least one anti-proliferative agent is formed by combining a composition comprising at least one anti-proliferative agent and a composition comprising at least one active agent. In some embodiments, the composition comprising at least one active agent is a composition described herein.

In some embodiments, when at least one anti-proliferative agent is present in the composition, the composition further comprising at least one anti-proliferative agent is formed by combining a composition comprising at least one anti-proliferative agent and a composition comprising at least one active agent prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 24 hours prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 6 hours prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent 15 seconds to 1 hour prior to use of the composition. In some embodiments, when at least one anti-proliferative agent is present in the composition, the composition further comprising at least one anti-proliferative agent is formed by combining a composition comprising at least one anti-proliferative agent and a composition comprising at least one active agent less than 2 hours prior to use.

In some embodiments, the at least one anti-proliferative agent is chosen from paclitaxel, paclitaxel derivatives, rapamycin, rapamycin derivatives, and pharmaceutically acceptable salts thereof. In some embodiments, the at least one anti-proliferative agent is paclitaxel. In some embodiments, the paclitaxel derivatives are chosen from docetaxel, and cabazitaxel. In some embodiments, the at least one anti-proliferative agent is rapamycin. In some embodiments, the rapamycin derivatives are chosen from everolimus, ridaforolimus, tacrolimus, umirolimus, and zotarolimus.

In some embodiments, when at least one anti-proliferative agent is present in the composition, the ratio of the volume of a composition comprising at least one anti-proliferative agent to the volume of a composition comprising at least one active agent ranges from 0.5:1 to 2.5:1 prior to combination. In some embodiments, when at least one anti-proliferative agent is present in the composition, the ratio of the volume of a composition comprising at least one anti-proliferative agent to the volume of a composition comprising at least one active agent ranges from 0.5:1 to 1.5:1 prior to combination. In some embodiments, when at least one anti-proliferative agent is present in the composition, the ratio of the volume of a composition comprising at least one anti-proliferative agent to the volume of a composition comprising at least one active agent ranges from 0.75:1 to 1.25:1 prior to combination.

In some embodiments, when at least one anti-proliferative agent is present in the composition, the ratio of the volume of a composition comprising at least one anti-proliferative agent to the volume of a composition comprising at least one active agent is 0.5:1 prior to combination. In some embodiments, when at least one anti-proliferative agent is present in the composition, the ratio of the volume of a composition comprising at least one anti-proliferative agent to the volume of a composition comprising at least one active agent is 0.75:1 prior to combination. In some embodiments, when at least one anti-proliferative agent is present in the composition, the ratio of the volume of a composition comprising at least one anti-proliferative agent to the volume of a composition comprising at least one active agent is 1:1 prior to combination. In some embodiments, when at least one anti-proliferative agent is present in the composition, the ratio of the volume of a composition comprising at least one anti-proliferative agent to the volume of a composition comprising at least one active agent is 1.25:1 prior to combination. In some embodiments, when at least one anti-proliferative agent is present in the composition, the ratio of the volume of a composition comprising at least one anti-proliferative agent to the volume of a composition comprising at least one active agent is 1.5:1 prior to combination. In some embodiments, when at least one anti-proliferative agent is present in the composition, the ratio of the volume of a composition comprising at least one anti-proliferative agent to the volume of a composition comprising at least one active agent is 1.75:1 prior to combination. In some embodiments, when at least one anti-proliferative agent is present in the composition, the ratio of the volume of a composition comprising at least one anti-proliferative agent to the volume of a composition comprising at least one active agent is 2:1 prior to combination. In some embodiments, when at least one anti-proliferative agent is present in the composition, the ratio of the volume of a composition comprising at least one anti-proliferative agent to the volume of a composition comprising at least one active agent is 2.25:1 prior to combination. In some embodiments, when at least one anti-proliferative agent is present in the composition, the ratio of the volume of a composition comprising at least one anti-proliferative agent to the volume of a composition comprising at least one active agent is 2.5:1 prior to combination.

In some embodiments, when at least one anti-proliferative agent is present in the composition, the molar ratio of the at least one anti-proliferative agent to the at least one active agent ranges from 0.25:4 to 4:0.25. In some embodiments, when at least one anti-proliferative agent is present in the composition, the molar ratio of the at least one anti-proliferative agent to the at least one active agent ranges from 0.75:2 to 2:0.75. In some embodiments, when at least one anti-proliferative agent is present in the composition, the molar ratio of the at least one anti-proliferative agent to the at least one active agent ranges from 0.75:1.5 to 1.25:1.5. In some embodiments, when at least one anti-proliferative agent is present in the composition, the molar ratio of the at least one anti-proliferative agent to the at least one active agent ranges from 0.9:1.3 to 1.1:1.5. In some embodiments, when at least one anti-proliferative agent is present in the composition, the molar ratio of the at least one anti-proliferative agent to the at least one active agent is 1:1.4.

Composition Properties

Composition pH

In some embodiments, the composition has a pH ranging from 4 to 8. In some embodiments, the composition has a pH ranging from 5 to 7. In some embodiments, the composition has a pH ranging from 5.5 to 6.5.

Without being bound by any theory, in some embodiments, a composition having a pH ranging from 5 to 7 may be suitable for intraarterial or intravenous delivery and/or may result in a composition where the at least one active agent does not degrade after a period of time compared to a composition having a pH outside of that range, as described herein below.

In some embodiments, the composition has a pH of 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, or 7. In some embodiments, the composition has a pH of 5. In some embodiments, the composition has a pH of 5.1. In some embodiments, the composition has a pH of 5.2. In some embodiments, the composition has a pH of 5.3. In some embodiments, the composition has a pH of 5.4. In some embodiments, the composition has a pH of 5.5. In some embodiments, the composition has a pH of 5.6. In some embodiments, the composition has a pH of 5.7. In some embodiments, the composition has a pH of 5.8. In some embodiments, the composition has a pH of 5.9. In some embodiments, the composition has a pH of 6. In some embodiments, the composition has a pH of 6.1. In some embodiments, the composition has a pH of 6.2. In some embodiments, the composition has a pH of 6.3. In some embodiments, the composition has a pH of 6.4. In some embodiments, the composition has a pH of 6.5. In some embodiments, the composition has a pH of 6.6. In some embodiments, the composition has a pH of 6.7. In some embodiments, the composition has a pH of 6.8. In some embodiments, the composition has a pH of 6.9. In some embodiments, the composition has a pH of 7.

In some embodiments, the pH of the composition remains constant or relatively constant for 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks after preparation of the composition.

Composition Stability

In some embodiments, provided herein is a composition comprising at least one active agent, wherein the at least one active agent does not degrade 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks after preparation of the composition. In some embodiments, degradation is determined by LC.

Without being bound by any theory, in some embodiments, a composition where the at least one active agent does not degrade for a specific period of time after preparation of the composition may be advantageous, for example, because (1) the concentration of the at least one active agent is predictable; and/or (2) degradation products of the at least one active agent may have deleterious effects if the composition is administered to a subject.

In some embodiments, the at least one active agent does not degrade 60 seconds after preparation of the composition. In some embodiments, the at least one active agent does not degrade 2 minutes after preparation of the composition. In some embodiments, the at least one active agent does not degrade 3 minutes after preparation of the composition. In some embodiments, the at least one active agent does not degrade 4 minutes after preparation of the composition. In some embodiments, the at least one active agent does not degrade 5 minutes after preparation of the composition. In some embodiments, degradation is determined by LC.

In some embodiments, the at least one active agent does not degrade 1 hour after preparation of the composition. In some embodiments, the at least one active agent does not degrade 2 hours after preparation of the composition. In some embodiments, the at least one active agent does not degrade 8 hours after preparation of the composition. In some embodiments, the at least one active agent does not degrade for 1 day after preparation of the composition. In some embodiments, degradation is determined by LC.

In some embodiments, the at least one active agent does not degrade 1 week after preparation of the composition. In some embodiments, the at least one active agent does not degrade 2 weeks after preparation of the composition. In some embodiments, the at least one active agent does not degrade 3 weeks after preparation of the composition. In some embodiments, the at least one active agent does not degrade 4 weeks after preparation of the composition. In some embodiments, the at least one active agent does not degrade 5 weeks after preparation of the composition. In some embodiments, the at least one active agent does not degrade 6 weeks after preparation of the composition. In some embodiments, the at least one active agent does not degrade 7 weeks after preparation of the composition. In some embodiments, the at least one active agent does not degrade 8 weeks after preparation of the composition. In some embodiments, the at least one active agent does not degrade 9 weeks after preparation of the composition. In some embodiments, the at least one active agent does not degrade 10 weeks after preparation of the composition. In some embodiments, the at least one active agent does not degrade 11 weeks after preparation of the composition. In some embodiments, the at least one active agent does not degrade 12 weeks after preparation of the composition. In some embodiments, degradation is determined by LC.

In some embodiments, the at least one active agent does not degrade 52 weeks after preparation of the composition. In some embodiments, the at least one active agent does not degrade 78 weeks after preparation of the composition. In some embodiments, the at least one active agent does not degrade 104 weeks after preparation of the composition. In some embodiments, the at least one active agent does not degrade 130 weeks after preparation of the composition. In some embodiments, the at least one active agent does not degrade 156 weeks after preparation of the composition. In some embodiments, the at least one active agent does not degrade 182 weeks after preparation of the composition. In some embodiments, the at least one active agent does not degrade 208 weeks after preparation of the composition. In some embodiments, degradation is determined by LC.

In some embodiments, the at least one active agent does not degrade 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, or 2 weeks after addition of at least one imaging agent to the composition. In some embodiments, degradation is determined by LC.

In some embodiments, the at least one active agent does not degrade 60 seconds after addition of at least one imaging agent to the composition. In some embodiments, the at least one active agent does not degrade 2 minutes after addition of at least one imaging agent to the composition. In some embodiments, the at least one active agent does not degrade 3 minutes after addition of at least one imaging agent to the composition. In some embodiments, the at least one active agent does not degrade 4 minutes after addition of at least one imaging agent to the composition. In some embodiments, the at least one active agent does not degrade 5 minutes after addition of at least one imaging agent to the composition. In some embodiments, the at least one active agent does not degrade 10 minutes after addition of at least one imaging agent to the composition. In some embodiments, the at least one active agent does not degrade 15 minutes after addition of at least one imaging agent to the composition. In some embodiments, the at least one active agent does not degrade 30 minutes after addition of at least one imaging agent to the composition. In some embodiments, the at least one active agent does not degrade 45 minutes after addition of at least one imaging agent to the composition. In some embodiments, the at least one active agent does not degrade 1 hour after addition of at least one imaging agent to the composition. In some embodiments, the at least one active agent does not degrade 8 hours after addition of at least one imaging agent to the composition. In some embodiments, the at least one active agent does not degrade 16 hours after addition of at least one imaging agent to the composition. In some embodiments, the at least one active agent does not degrade 24 hours after addition of at least one imaging agent to the composition. In some embodiments, the at least one active agent does not degrade 2 days after addition of at least one imaging agent to the composition. In some embodiments, the at least one active agent does not degrade 3 days after addition of at least one imaging agent to the composition. In some embodiments, the at least one active agent does not degrade 1 week after addition of at least one imaging agent to the composition. In some embodiments, the at least one active agent does not degrade 2 weeks after addition of at least one imaging agent to the composition. In some embodiments, the at least one active agent does not degrade 3 weeks after addition of at least one imaging agent to the composition. In some embodiments, the at least one active agent does not degrade 1 month after addition of at least one imaging agent to the composition. In some embodiments, degradation is determined by LC.

In some embodiments, the at least one active agent does not degrade 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, or 2 weeks after combining with a composition comprising at least one anti-proliferative agent. In some embodiments, degradation is determined by LC.

In some embodiments, the at least one active agent does not degrade 60 seconds after combining with a composition comprising at least one anti-proliferative agent. In some embodiments, the at least one active agent does not degrade 2 minutes after combining with a composition comprising at least one anti-proliferative agent. In some embodiments, the at least one active agent does not degrade 3 minutes after combining with a composition comprising at least one anti-proliferative agent. In some embodiments, the at least one active agent does not degrade 4 minutes after combining with a composition comprising at least one anti-proliferative agent. In some embodiments, the at least one active agent does not degrade 5 minutes after combining with a composition comprising at least one anti-proliferative agent. In some embodiments, the at least one active agent does not degrade 10 minutes after combining with a composition comprising at least one anti-proliferative agent. In some embodiments, the at least one active agent does not degrade 15 minutes after combining with a composition comprising at least one anti-proliferative agent. In some embodiments, the at least one active agent does not degrade 30 minutes after combining with a composition comprising at least one anti-proliferative agent. In some embodiments, the at least one active agent does not degrade 45 minutes after combining with a composition comprising at least one anti-proliferative agent. In some embodiments, the at least one active agent does not degrade 1 hour after combining with a composition comprising at least one anti-proliferative agent. In some embodiments, the at least one active agent does not degrade 8 hours after combining with a composition comprising at least one anti-proliferative agent. In some embodiments, the at least one active agent does not degrade 16 hours after combining with a composition comprising at least one anti-proliferative agent. In some embodiments, the at least one active agent does not degrade 24 hours after combining with a composition comprising at least one anti-proliferative agent. In some embodiments, the at least one active agent does not degrade 2 days after combining with a composition comprising at least one anti-proliferative agent. In some embodiments, the at least one active agent does not degrade 3 days after combining with a composition comprising at least one anti-proliferative agent. In some embodiments, the at least one active agent does not degrade 1 week after combining with a composition comprising at least one anti-proliferative agent. In some embodiments, the at least one active agent does not degrade 2 weeks after combining with a composition comprising at least one anti-proliferative agent. In some embodiments, the at least one active agent does not degrade 3 weeks after combining with a composition comprising at least one anti-proliferative agent. In some embodiments, the at least one active agent does not degrade 1 month after combining with a composition comprising at least one anti-proliferative agent. In some embodiments, degradation is determined by LC.

In some embodiments, the at least one anti-proliferative agent does not degrade 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, or 2 weeks after combining a composition comprising at least one active agent with a composition comprising at least one anti-proliferative agent. In some embodiments, degradation is determined by LC.

In some embodiments, the at least one anti-proliferative agent does not degrade 60 seconds after combining a composition comprising at least one active agent with a composition comprising at least one anti-proliferative agent. In some embodiments, the at least one anti-proliferative agent does not degrade 2 minutes after combining a composition comprising at least one active agent with a composition comprising at least one anti-proliferative agent. In some embodiments, the at least one anti-proliferative agent does not degrade 3 minutes after combining a composition comprising at least one active agent with a composition comprising at least one anti-proliferative agent. In some embodiments, the at least one anti-proliferative agent does not degrade 4 minutes after combining a composition comprising at least one active agent with a composition comprising at least one anti-proliferative agent. In some embodiments, the at least one anti-proliferative agent does not degrade 5 minutes after combining a composition comprising at least one active agent with a composition comprising at least one anti-proliferative agent.

In some embodiments, the at least one anti-proliferative agent does not degrade 10 minutes after combining a composition comprising at least one active agent with a composition comprising at least one anti-proliferative agent. In some embodiments, the at least one anti-proliferative agent does not degrade 15 minutes after combining a composition comprising at least one active agent with a composition comprising at least one anti-proliferative agent. In some embodiments, the at least one anti-proliferative agent does not degrade 30 minutes after combining a composition comprising at least one active agent with a composition comprising at least one anti-proliferative agent. In some embodiments, the at least one anti-proliferative agent does not degrade 45 minutes after combining a composition comprising at least one active agent with a composition comprising at least one anti-proliferative agent.

In some embodiments, the at least one anti-proliferative agent does not degrade 1 hour after combining a composition comprising at least one active agent with a composition comprising at least one anti-proliferative agent. In some embodiments, the at least one anti-proliferative agent does not degrade 8 hours after combining a composition comprising at least one active agent with a composition comprising at least one anti-proliferative agent. In some embodiments, the at least one anti-proliferative agent does not degrade 16 hours after combining a composition comprising at least one active agent with a composition comprising at least one anti-proliferative agent. In some embodiments, the at least one anti-proliferative agent does not degrade 24 hours after combining a composition comprising at least one active agent with a composition comprising at least one anti-proliferative agent.

In some embodiments, the at least one anti-proliferative agent does not degrade 2 days after combining a composition comprising at least one active agent with a composition comprising at least one anti-proliferative agent. In some embodiments, the at least one anti-proliferative agent does not degrade 3 days after combining a composition comprising at least one active agent with a composition comprising at least one anti-proliferative agent.

In some embodiments, the at least one anti-proliferative agent does not degrade 1 week after combining a composition comprising at least one active agent with a composition comprising at least one anti-proliferative agent. In some embodiments, the at least one anti-proliferative agent does not degrade 2 weeks after combining a composition comprising at least one active agent with a composition comprising at least one anti-proliferative agent. In some embodiments, the at least one anti-proliferative agent does not degrade 3 weeks after combining a composition comprising at least one active agent with a composition comprising at least one anti-proliferative agent. In some embodiments, the at least one anti-proliferative agent does not degrade 1 month after combining a composition comprising at least one active agent with a composition comprising at least one anti-proliferative agent. In some embodiments, degradation is determined by LC.

In some embodiments, the at least one active agent does not degrade 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, or 2 weeks after combining with a composition comprising at least one anti-proliferative agent and a composition comprising at least one imaging agent. In some embodiments, degradation is determined by LC.

In some embodiments, the at least one active agent does not degrade 60 seconds after combining with a composition comprising at least one anti-proliferative agent and a composition comprising at least one imaging agent. In some embodiments, the at least one active agent does not degrade 2 minutes after combining with a composition comprising at least one anti-proliferative agent and a composition comprising at least one imaging agent. In some embodiments, the at least one active agent does not degrade 3 minutes after combining with a composition comprising at least one anti-proliferative agent and a composition comprising at least one imaging agent. In some embodiments, the at least one active agent does not degrade 4 minutes after combining with a composition comprising at least one anti-proliferative agent and a composition comprising at least one imaging agent.

In some embodiments, the at least one active agent does not degrade 5 minutes after combining with a composition comprising at least one anti-proliferative agent and a composition comprising at least one imaging agent. In some embodiments, the at least one active agent does not degrade 10 minutes after combining with a composition comprising at least one anti-proliferative agent and a composition comprising at least one imaging agent. In some embodiments, the at least one active agent does not degrade 15 minutes after combining with a composition comprising at least one anti-proliferative agent and a composition comprising at least one imaging agent. In some embodiments, the at least one active agent does not degrade 30 minutes after combining with a composition comprising at least one anti-proliferative agent and a composition comprising at least one imaging agent.

In some embodiments, the at least one active agent does not degrade 45 minutes after combining with a composition comprising at least one anti-proliferative agent and a composition comprising at least one imaging agent. In some embodiments, the at least one active agent does not degrade 1 hour after combining with a composition comprising at least one anti-proliferative agent and a composition comprising at least one imaging agent. In some embodiments, the at least one active agent does not degrade 8 hours after combining with a composition comprising at least one anti-proliferative agent and a composition comprising at least one imaging agent.

In some embodiments, the at least one active agent does not degrade 16 hours after combining with a composition comprising at least one anti-proliferative agent and a composition comprising at least one imaging agent. In some embodiments, the at least one active agent does not degrade 24 hours after combining with a composition comprising at least one anti-proliferative agent and a composition comprising at least one imaging agent. In some embodiments, the at least one active agent does not degrade 2 days after combining with a composition comprising at least one anti-proliferative agent and a composition comprising at least one imaging agent. In some embodiments, the at least one active agent does not degrade 3 days after combining with a composition comprising at least one anti-proliferative agent and a composition comprising at least one imaging agent.

In some embodiments, the at least one active agent does not degrade 1 week after combining with a composition comprising at least one anti-proliferative agent and a composition comprising at least one imaging agent. In some embodiments, the at least one active agent does not degrade 2 weeks after combining with a composition comprising at least one anti-proliferative agent and a composition comprising at least one imaging agent. In some embodiments, the at least one active agent does not degrade 3 weeks after combining with a composition comprising at least one anti-proliferative agent and a composition comprising at least one imaging agent. In some embodiments, the at least one active agent does not degrade 1 month after combining with a composition comprising at least one anti-proliferative agent and a composition comprising at least one imaging agent. In some embodiments, degradation is determined by LC.

In some embodiments, the at least one anti-proliferative agent does not degrade 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, or 2 weeks after combining a composition comprising at least one active agent with a composition comprising at least one anti-proliferative agent and a composition comprising at least one imaging agent. In some embodiments, degradation is determined by LC.

In some embodiments, the at least one anti-proliferative agent does not degrade 60 seconds after combining a composition comprising at least one active agent with a composition comprising at least one anti-proliferative agent and a composition comprising at least one imaging agent. In some embodiments, the at least one anti-proliferative agent does not degrade 2 minutes after combining a composition comprising at least one active agent with a composition comprising at least one anti-proliferative agent and a composition comprising at least one imaging agent.

In some embodiments, the at least one anti-proliferative agent does not degrade 3 minutes after combining a composition comprising at least one active agent with a composition comprising at least one anti-proliferative agent and a composition comprising at least one imaging agent. In some embodiments, the at least one anti-proliferative agent does not degrade 4 minutes after combining a composition comprising at least one active agent with a composition comprising at least one anti-proliferative agent and a composition comprising at least one imaging agent. In some embodiments, the at least one anti-proliferative agent does not degrade 5 minutes after combining a composition comprising at least one active agent with a composition comprising at least one anti-proliferative agent and a composition comprising at least one imaging agent.

In some embodiments, the at least one anti-proliferative agent does not degrade 10 minutes after combining a composition comprising at least one active agent with a composition comprising at least one anti-proliferative agent and a composition comprising at least one imaging agent. In some embodiments, the at least one anti-proliferative agent does not degrade 15 minutes after combining a composition comprising at least one active agent with a composition comprising at least one anti-proliferative agent and a composition comprising at least one imaging agent. In some embodiments, the at least one anti-proliferative agent does not degrade 30 minutes after combining a composition comprising at least one active agent with a composition comprising at least one anti-proliferative agent and a composition comprising at least one imaging agent.

In some embodiments, the at least one anti-proliferative agent does not degrade 45 minutes after combining a composition comprising at least one active agent with a composition comprising at least one anti-proliferative agent and a composition comprising at least one imaging agent. In some embodiments, the at least one anti-proliferative agent does not degrade 1 hour after combining a composition comprising at least one active agent with a composition comprising at least one anti-proliferative agent and a composition comprising at least one imaging agent. In some embodiments, the at least one anti-proliferative agent does not degrade 8 hours after combining a composition comprising at least one active agent with a composition comprising at least one anti-proliferative agent and a composition comprising at least one imaging agent.

In some embodiments, the at least one anti-proliferative agent does not degrade 16 hours after combining a composition comprising at least one active agent with a composition comprising at least one anti-proliferative agent and a composition comprising at least one imaging agent. In some embodiments, the at least one anti-proliferative agent does not degrade 24 hours after combining a composition comprising at least one active agent with a composition comprising at least one anti-proliferative agent and a composition comprising at least one imaging agent. In some embodiments, the at least one anti-proliferative agent does not degrade 2 days after combining a composition comprising at least one active agent with a composition comprising at least one anti-proliferative agent and a composition comprising at least one imaging agent.

In some embodiments, the at least one anti-proliferative agent does not degrade 3 days after combining a composition comprising at least one active agent with a composition comprising at least one anti-proliferative agent and a composition comprising at least one imaging agent. In some embodiments, the at least one anti-proliferative agent does not degrade 1 week after combining a composition comprising at least one active agent with a composition comprising at least one anti-proliferative agent and a composition comprising at least one imaging agent. In some embodiments, the at least one anti-proliferative agent does not degrade 2 weeks after combining a composition comprising at least one active agent with a composition comprising at least one anti-proliferative agent and a composition comprising at least one imaging agent.

In some embodiments, the at least one anti-proliferative agent does not degrade 3 weeks after combining a composition comprising at least one active agent with a composition comprising at least one anti-proliferative agent and a composition comprising at least one imaging agent. In some embodiments, the at least one anti-proliferative agent does not degrade 1 month after combining a composition comprising at least one active agent with a composition comprising at least one anti-proliferative agent and a composition comprising at least one imaging agent. In some embodiments, degradation is determined by LC.

In some embodiments, the at least one imaging agent does not degrade 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, or 2 weeks after combining a composition comprising at least one active agent with a composition comprising at least one anti-proliferative agent and a composition comprising at least one imaging agent. In some embodiments, degradation is determined by LC.

In some embodiments, the at least one imaging agent does not degrade 60 seconds after combining a composition comprising at least one active agent with a composition comprising at least one anti-proliferative agent and a composition comprising at least one imaging agent. In some embodiments, the at least one imaging agent does not degrade 2 minutes after combining a composition comprising at least one active agent with a composition comprising at least one anti-proliferative agent and a composition comprising at least one imaging agent.

In some embodiments, the at least one imaging agent does not degrade 3 minutes after combining a composition comprising at least one active agent with a composition comprising at least one anti-proliferative agent and a composition comprising at least one imaging agent. In some embodiments, the at least one imaging agent does not degrade 4 minutes after combining a composition comprising at least one active agent with a composition comprising at least one anti-proliferative agent and a composition comprising at least one imaging agent. In some embodiments, the at least one imaging agent does not degrade 5 minutes after combining a composition comprising at least one active agent with a composition comprising at least one anti-proliferative agent and a composition comprising at least one imaging agent.

In some embodiments, the at least one imaging agent does not degrade 10 minutes after combining a composition comprising at least one active agent with a composition comprising at least one anti-proliferative agent and a composition comprising at least one imaging agent. In some embodiments, the at least one imaging agent does not degrade 15 minutes after combining a composition comprising at least one active agent with a composition comprising at least one anti-proliferative agent and a composition comprising at least one imaging agent.

In some embodiments, the at least one imaging agent does not degrade 30 minutes after combining a composition comprising at least one active agent with a composition comprising at least one anti-proliferative agent and a composition comprising at least one imaging agent. In some embodiments, the at least one imaging agent does not degrade 45 minutes after combining a composition comprising at least one active agent with a composition comprising at least one anti-proliferative agent and a composition comprising at least one imaging agent. In some embodiments, the at least one imaging agent does not degrade 1 hour after combining a composition comprising at least one active agent with a composition comprising at least one anti-proliferative agent and a composition comprising at least one imaging agent. In some embodiments, the at least one imaging agent does not degrade 8 hours after combining a composition comprising at least one active agent with a composition comprising at least one anti-proliferative agent and a composition comprising at least one imaging agent.

In some embodiments, the at least one imaging agent does not degrade 16 hours after combining a composition comprising at least one active agent with a composition comprising at least one anti-proliferative agent and a composition comprising at least one imaging agent. In some embodiments, the at least one imaging agent does not degrade 24 hours after combining a composition comprising at least one active agent with a composition comprising at least one anti-proliferative agent and a composition comprising at least one imaging agent. In some embodiments, the at least one imaging agent does not degrade 2 days after combining a composition comprising at least one active agent with a composition comprising at least one anti-proliferative agent and a composition comprising at least one imaging agent.

In some embodiments, the at least one imaging agent does not degrade 3 days after combining a composition comprising at least one active agent with a composition comprising at least one anti-proliferative agent and a composition comprising at least one imaging agent. In some embodiments, the at least one imaging agent does not degrade 1 week after combining a composition comprising at least one active agent with a composition comprising at least one anti-proliferative agent and a composition comprising at least one imaging agent. In some embodiments, the at least one imaging agent does not degrade 2 weeks after combining a composition comprising at least one active agent with a composition comprising at least one anti-proliferative agent and a composition comprising at least one imaging agent.

In some embodiments, the at least one imaging agent does not degrade 3 weeks after combining a composition comprising at least one active agent with a composition comprising at least one anti-proliferative agent and a composition comprising at least one imaging agent. In some embodiments, the at least one imaging agent does not degrade 1 month after combining a composition comprising at least one active agent with a composition comprising at least one anti-proliferative agent and a composition comprising at least one imaging agent. In some embodiments, degradation is determined by LC.

In some embodiments, there is 0% to less than 15%, less than 14%, less than 13%, less than 12%, less than 11%, less than 10%, less than 9%, less than 8%, less than 7%, less than 6%, less than 5%, less than 4.5%, less than 4%, less than 3.5%, less than 3%, less than 2.5%, less than 2%, less than 1.5%, less than 1%, less than 0.75%, less than 0.5%, less than 0.45%, less than 0.4%, less than 0.35%, less than 0.3%, less than 0.25%, less than 0.2%, less than 0.15%, less than 0.1%, or less than 0.05% by weight of impurities present in the composition 1 week, 2 weeks, 1 month, 2 months, 3 months, 4 months, 5 months, 6 months, 9 months, 12 months, 18 months, 24 months, 30 months, or 36 months after preparation of the composition.

In some embodiments, the composition further comprises at least one imaging agent. In some embodiments, the composition further comprises at least one anti-proliferative agent. In some embodiments, the composition further comprises at least one imaging agent and at least one anti-proliferative agent. In some embodiments, the amount of impurities is determined by LC.

In some embodiments, there is 0% to less than 5% by weight of impurities present in the composition 24 hours after preparation of the composition. In some embodiments, there is 0% to less than 2.5% by weight of impurities present in the composition 24 hours after preparation of the composition. In some embodiments, there is 0% to less than 1% by weight of impurities present in the composition 24 hours after preparation of the composition. In some embodiments, there is 0% to less than 0.5% by weight of impurities present in the composition 24 hours after preparation of the composition.

In some embodiments, there is 0% to less than 0.25% by weight of impurities present in the composition 24 hours after preparation of the composition. In some embodiments, there is 0% to less than 0.05% by weight of impurities present in the composition 24 hours after preparation of the composition. In some embodiments, the amount of impurities is determined by LC. In some embodiments, the composition further comprises at least one imaging agent. In some embodiments, the composition further comprises at least one anti-proliferative agent. In some embodiments, the composition further comprises at least one imaging agent and at least one anti-proliferative agent.

In some embodiments, there is 0% to less than 5% by weight of impurities present in the composition 2 months after preparation of the composition. In some embodiments, there is 0% to less than 2.5% by weight of impurities present in the composition 2 months after preparation of the composition. In some embodiments, there is 0% to less than 1% by weight of impurities present in the composition 2 months after preparation of the composition. In some embodiments, there is 0% to less than 0.5% by weight of impurities present in the composition 2 months after preparation of the composition. In some embodiments, there is 0% to less than 0.25% by weight of impurities present in the composition 2 months after preparation of the composition. In some embodiments, there is 0% to less than 0.05% by weight of impurities present in the composition 2 months after preparation of the composition. In some embodiments, the amount of impurities is determined by LC. In some embodiments, the composition further comprises at least one imaging agent. In some embodiments, the composition further comprises at least one anti-proliferative agent. In some embodiments, the composition further comprises at least one imaging agent and at least one anti-proliferative agent.

In some embodiments, there is 0% to less than 5% by weight of impurities present in the composition 12 months after preparation of the composition. In some embodiments, there is 0% to less than 2.5% by weight of impurities present in the composition 12 months after preparation of the composition. In some embodiments, there is 0% to less than 1% by weight of impurities present in the composition 12 months after preparation of the composition. In some embodiments, there is 0% to less than 0.5% by weight of impurities present in the composition 12 months after preparation of the composition. In some embodiments, there is 0% to less than 0.25% by weight of impurities present in the composition 12 months after preparation of the composition. In some embodiments, there is 0% to less than 0.05% by weight of impurities present in the composition 12 months after preparation of the composition. In some embodiments, the amount of impurities is determined by LC. In some embodiments, the composition further comprises at least one imaging agent. In some embodiments, the composition further comprises at least one anti-proliferative agent. In some embodiments, the composition further comprises at least one imaging agent and at least one anti-proliferative agent.

In some embodiments, there is 0% to less than 5% by weight of impurities present in the composition 36 months after preparation of the composition. In some embodiments, there is 0% to less than 2.5% by weight of impurities present in the composition 36 months after preparation of the composition. In some embodiments, there is 0% to less than 1% by weight of impurities present in the composition 36 months after preparation of the composition. In some embodiments, there is 0% to less than 0.5% by weight of impurities present in the composition 36 months after preparation of the composition. In some embodiments, there is 0% to less than 0.25% by weight of impurities present in the composition 36 months after preparation of the composition. In some embodiments, there is 0% to less than 0.15% by weight of impurities preset in the composition 36 months after preparation of the composition. In some embodiments, there is 0% to less than 0.05% by weight of impurities present in the composition 36 months after preparation of the composition. In some embodiments, the amount of impurities is determined by LC. In some embodiments, the composition further comprises at least one imaging agent. In some embodiments, the composition further comprises at least one anti-proliferative agent. In some embodiments, the composition further comprises at least one imaging agent and at least one anti-proliferative agent.

In some embodiments, provided herein is a composition, wherein the total amount of impurities in the composition 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks after preparation of the composition is less than five-fold the total amount of impurities initially present in the composition. In some embodiments, the amount of impurities is determined by LC. In some embodiments, the composition further comprises at least one imaging agent. In some embodiments, the composition further comprises at least one anti-proliferative agent. In some embodiments, the composition further comprises at least one imaging agent and at least one anti-proliferative agent.

In some embodiments, provided herein is a composition, wherein the total amount of impurities in the composition 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks after preparation of the composition is less than four-fold the total amount of impurities initially present in the composition. In some embodiments, the amount of impurities is determined by LC. In some embodiments, the composition further comprises at least one imaging agent. In some embodiments, the composition further comprises at least one anti-proliferative agent. In some embodiments, the composition further comprises at least one imaging agent and at least one anti-proliferative agent.

In some embodiments, provided herein is a composition, wherein the total amount of impurities in the composition 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks after preparation of the composition is less than three-fold the total amount of impurities initially present in the composition. In some embodiments, the amount of impurities is determined by LC. In some embodiments, the composition further comprises at least one imaging agent. In some embodiments, the composition further comprises at least one anti-proliferative agent. In some embodiments, the composition further comprises at least one imaging agent and at least one anti-proliferative agent.

In some embodiments, provided herein is a composition, wherein the total amount of impurities in the composition 15 seconds, 30 seconds, 45 seconds, 60 seconds, 90 seconds, 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 12 hours, 18 hours, 24 hours, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks after preparation of the composition is less than two-fold the total amount of impurities initially present in the composition. In some embodiments, the amount of impurities is determined by LC. In some embodiments, the composition further comprises at least one imaging agent. In some embodiments, the composition further comprises at least one anti-proliferative agent. In some embodiments, the composition further comprises at least one imaging agent and at least one anti-proliferative agent.

In some embodiments, the composition can be stored at a temperature ranging from 1° C. to 40° C. for 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks after preparation of the composition. In some embodiments, the at least one active agent does not degrade during storage. In some embodiments, the total amount of impurities in the composition is less than five-fold, less than four-fold, less than three-fold, or less than two-fold the total amount of impurities initially present in the composition before storage. In some embodiments, 0% to less than 15%, to less than 10%, to less than 5%, to less than 2%, to less than 1%, to less than 0.5%, to less than 0.25%, to less than 0.1%, or to less than 0.05% by weight of impurities are present in the composition after storage. In some embodiments, storage does not affect the percentage of impurities present in the composition. In some embodiments the composition can be stored at 5° C., at 25° C., or at 40° C. In some embodiments, the composition can be stored at a temperature from 25° C. to 40° C.

### NON-LIMITING EXEMPLARY COMPOSITION EMBODIMENTS

In some embodiments, provided herein is a composition comprising at least one active agent; at least one tonicity agent; at least one buffer; and at least one vehicle.

In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle. In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle, wherein the composition has a pH of 6. In some embodiments, the composition comprises at least one active agent in an amount ranging from 0.01% to 1% by weight of the composition; at least one tonicity agent in an amount ranging from 0.5% to 1.5% by weight of the composition; at least one buffer in an amount ranging from 0.05% to 0.4% by weight of the composition; and at least one vehicle, wherein the composition has a pH of 6, and wherein the at least one active agent does not degrade 156 weeks after preparation of the composition.

In some embodiments, provided herein is a composition comprising at least one active agent chosen from Compound of Formula (I) and pharmaceutically acceptable salts thereof; at least one tonicity agent chosen from potassium chloride and sodium chloride; at least one buffer chosen from potassium phosphate and sodium phosphate; and at least one vehicle comprising water. In some embodiments, the composition has a pH of 6. In some embodiments, provided herein is a composition comprising at least one active agent chosen from Compound of Formula (I) and pharmaceutically acceptable salts thereof; at least one tonicity agent chosen from potassium chloride and sodium chloride; at least one buffer chosen from potassium phosphate and sodium phosphate; and at least one vehicle comprising water, wherein the composition has a pH of 6, and wherein the at least one active agent does not degrade 156 weeks after preparation of the composition.

In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water. In some embodiments, the composition further comprises at least one active agent. In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water, wherein the composition has a pH of 6. In some embodiments, the composition further comprises at least one active agent. In some embodiments, provided herein is a composition comprising at least one active agent chosen from diacetate salts of Compound of Formula (I); at least one tonicity agent chosen from potassium chloride and sodium chloride; at least one buffer chosen from potassium phosphate and sodium phosphate; and at least one vehicle comprising water, wherein the composition has a pH of 6, and wherein the at least one active agent does not degrade 156 weeks after preparation of the composition. In some embodiments, degradation is determined by LC. In some embodiments, the composition further comprises at least one active agent. In some embodiments, the composition further comprises at least one anti-proliferative agent.

In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, wherein the composition has a pH of 6. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, wherein the composition has a pH of 6, and wherein the at least one active agent does not degrade 156 weeks after preparation of the composition. In some embodiments, degradation is determined by LC. In some embodiments, the composition further comprises at least one active agent. In some embodiments, the composition further comprises at least one anti-proliferative agent.

In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent. In some embodiments, the composition further comprises at least one anti-proliferative agent. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent. In some embodiments, the composition further comprises at least one anti-proliferative agent. In some embodiments, the composition further comprises at least one active agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent, wherein the at least one imaging agent comprises a radiographic contrast agent. In some embodiments, the composition further comprises at least one anti-proliferative agent. In some embodiments, provided herein is a composition comprising at least one active agent chosen from diacetate salts of Compound of Formula (I) in an amount ranging from 0.01% to 1% by weight of the composition; at least one tonicity agent chosen from potassium chloride and sodium chloride in an amount ranging from 0.5% to 1.5% by weight of the composition; at least one buffer chosen from potassium phosphate and sodium phosphate in an amount ranging from 0.05% to 0.4% by weight of the composition; and at least one vehicle comprising water, further comprising at least one imaging agent, wherein the at least one active agent does not degrade 24 hours after preparation of the composition. In some embodiments, degradation is determined by LC. In some embodiments, the composition further comprises at least one active agent. In some embodiments, the composition further comprises at least one anti-proliferative agent.

In some embodiments, the at least one active agent is chosen from a Compound of Formula (I) and pharmaceutically acceptable salts thereof. In some embodiments, the at least one active agent is a diacetate salt of a Compound of Formula (I).

In some embodiments, the at least one active agent is a diacetate salt of a Compound of Formula (I) present in an amount of 0.01% to 1% by weight of the composition. In some embodiments, the at least one active agent is a diacetate salt of a Compound of Formula (I) present in an amount of 0.01% to 1% by weight of the composition and the at least one tonicity agent is chosen from potassium chloride and sodium chloride. In some embodiments, the at least one active agent is a diacetate salt of a Compound of Formula (I) present in an amount of 0.01% to 1% by weight of the composition and the at least one tonicity agent is chosen from potassium chloride and sodium chloride present in an amount ranging from 0.5% to 1.5% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; and the at least one buffer is chosen from sodium phosphate dibasic and potassium phosphate monobasic. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; and the at least one buffer is chosen from sodium phosphate dibasic and potassium phosphate monobasic present in an amount of 0.1% to 0.16% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, wherein the composition has a pH of 6.

In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, wherein the pH of the composition is 6.

In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose in an amount ranging from 0.1% to 0.3% by weight of the composition. In some embodiments, the at least one active agent is a diacetate salt of a Compound of Formula (I) present in an amount of 0.01% to 1% by weight of the composition; the at least one tonicity agent is chosen from potassium chloride and sodium chloride present in an amount ranging from 0.5% to 1.5% by weight of the composition; the at least one buffer is chosen from sodium phosphate dibasic and potassium phosphate monobasic present in an amount of 0.1% to 0.16% by weight of the composition; and the at least one vehicle is water, and the composition further comprises at least one pH modulation agent chosen from acetic acid and sodium hydroxide, and at least one viscosity agent chosen from methylcellulose, sodium carboxymethylcellulose, and hydroxypropyl cellulose in an amount ranging from 0.1% to 0.3% by weight of the composition, wherein the composition has a pH of 6.

### Additional Embodiments

Embodiment 1. A composition comprising:

at least one active agent chosen from a Compound of Formula (I):

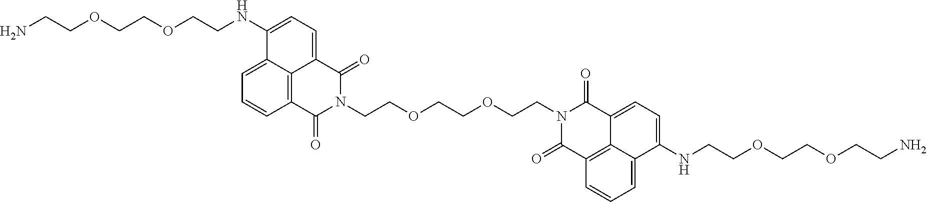 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]

(I) and pharmaceutically acceptable salts thereof;
at least one tonicity agent;
at least one buffer; and
at least one vehicle.

Embodiment 2. The composition according to embodiment 1, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate.

Embodiment 3. The composition according to embodiment 1 or 2, wherein the at least one active agent is present in an amount ranging from 0.01% to 2.5% by weight of the composition.

Embodiment 4. The composition according to any one of embodiments 1 to 3, wherein the at least one active agent is present in an amount of 0.01%, 0.02%, 0.03%, 0.04%, 0.05%, 0.06%, 0.07%, 0.08%, 0.09%, 0.1%, 0.12%, 0.14%, 0.16%, 0.18%, 0.2%, 0.22%, 0.24%, 0.26%, 0.28%, 0.3%, 0.32%, 0.34%, 0.36%, 0.38%, 0.4%, 0.42%, 0.44%, 0.46%, 0.48%, 0.5%, 0.52%, 0.54%, or 0.56% by weight of the composition.

Embodiment 5. The composition according to any one of embodiments 1 to 4, wherein the at least one active agent is present in an amount of 0.1%, 0.11%, 0.12%, 0.13%, 0.14%, 0.15%, 0.16%, 0.17%, 0.18%, 0.19%, 0.2%, 0.21%, 0.22%, 0.23%, 0.24%, 0.25%, 0.26%, 0.27%, 0.28%, 0.29%, 0.3%, 0.31%, 0.32%, 0.33%, 0.34%, 0.35%, 0.36%, 0.37%, 0.38%, 0.39%, 0.4%, 0.41%, 0.42%, 0.43%, 0.44%, 0.45%, 0.46%, 0.47%, 0.48%, 0.49%, or 0.5% by weight of the composition.

Embodiment 6. The composition according to any one of embodiments 1 to 5, wherein the at least one tonicity agent is chosen from dextrose, sorbitol, lactose, mannitol, sodium chloride, potassium chloride, and glycerol.

Embodiment 7. The composition according to any one of embodiments 1 to 6, wherein the at least one tonicity agent is sodium chloride.

Embodiment 8. The composition according to any one of embodiments 1 to 6, wherein the at least one tonicity agent is potassium chloride.

Embodiment 9. The composition according to any one of embodiments 1 to 8, wherein the at least one tonicity agent is present in an amount up to 2% by weight of the composition.

Embodiment 10. The composition according to any one of embodiments 1 to 9, wherein the at least one tonicity agent is present in an amount of 0.5% to 1.5% by weight of the composition.

Embodiment 11. The composition according to any one of embodiments 1 to 10, wherein the at least one tonicity agent is present in an amount of 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, or 0.9% by weight of the composition.

Embodiment 12. The composition according to any one of embodiments 1 to 11, wherein the at least one tonicity agent is present in an amount of 0.72%, 0.74%, 0.76%, 0.78%, 0.8%, 0.82%, or 0.84% by weight of the composition.

Embodiment 13. The composition according to any one of embodiments 1 to 12, wherein the at least one buffer is chosen from potassium phosphate, sodium phosphate, potassium citrate, sodium citrate, potassium acetate, sodium acetate, potassium lactate, sodium lactate, potassium tartrate, maleic acid, and sodium tartrate.

Embodiment 14. The composition according to embodiment 13, wherein the potassium phosphate is chosen from potassium phosphate monobasic and potassium phosphate dibasic.

Embodiment 15. The composition according to embodiment 13, wherein the sodium phosphate is chosen from sodium phosphate monobasic and sodium phosphate dibasic.

Embodiment 16. The composition according to any one of embodiments 1 to 15, wherein the at least one buffer is present in an amount up to 1% by weight of the composition.

Embodiment 17. The composition according to any one of embodiments 1 to 16, wherein the at least one buffer is present in an amount ranging from 0.05% to 0.4% by weight of the composition.

Embodiment 18. The composition according to any one of embodiments 1 to 17, wherein the at least one buffer is present in an amount of 0.06%, 0.08%, 0.1%, 0.12%, 0.14%, 0.16%, 0.18%, 0.2%, 0.22%, 0.24%, 0.26%, 0.28%, or 0.3% by weight of the composition.

Embodiment 19. The composition according to any one of embodiments 1 to 17, wherein the at least one buffer is present in an amount of 0.12% to 0.16% by weight of the composition.

Embodiment 20. The composition according to any one of embodiments 1 to 17, wherein the at least one buffer comprises sodium phosphate in an amount ranging from 0.01% to 0.03% by weight of the composition and potassium phosphate in an amount ranging from 0.1% to 0.14% by weight of the composition.

Embodiment 21. The composition according to embodiment 20, wherein the sodium phosphate is sodium phosphate dibasic.

Embodiment 22. The composition according to embodiment 20, wherein the potassium phosphate is potassium phosphate monobasic.

Embodiment 23. The composition according to any one of embodiments 1 to 22, wherein the at least one vehicle is chosen from water, ethanol, isopropanol, polyethylene glycols, and propylene glycols.

Embodiment 24. The composition according to embodiment 23, wherein the polyethylene glycols are chosen from polyethylene glycol 300, polyethylene glycol 400, and polyethylene glycol 600.

Embodiment 25. The composition according to any one of embodiments 1 to 24, wherein the at least one vehicle is water.

Embodiment 26. The composition according to any one of embodiments 1 to 25, further comprising at least one pH modulation agent.

Embodiment 27. The composition according to embodiment 26, wherein the at least one pH modulation agent is chosen from acetic acid, carbonic acid, citric acid, sodium bicarbonate, and sodium hydroxide.

Embodiment 28. The composition according to embodiment 26 or 27, wherein the at least one pH modulation agent comprises acetic acid.

Embodiment 29. The composition according to any one of embodiments 26 to 28, wherein the at least one pH modulation agent comprises sodium hydroxide.

Embodiment 30. The composition according to any one of embodiments 1 to 29, further comprising at least one viscosity agent.

Embodiment 31. The composition according to embodiment 30, wherein the at least one viscosity agent is chosen from gelatin, methylcellulose, hydroxypropyl cellulose, hydroxypropyl methylcellulose, hydroxyethyl cellulose, hydroxypropylmethyl cellulose, methylhydroxyethyl cellulose, methylhydroxypropyl cellulose, hydroxyethylcarboxymethyl cellulose, carboxymethyl cellulose, carboxymethylhydroxyethyl cellulose, and sodium carboxymethylcellulose.

Embodiment 32. The composition according to embodiment 31, wherein the at least one viscosity agent is methyl cellulose.

Embodiment 33. The composition according to embodiment 31, wherein the at least one viscosity agent is sodium carboxymethyl cellulose.

Embodiment 34. The composition according to embodiment 31, wherein the at least one viscosity agent is hydroxypropyl cellulose.

Embodiment 35. The composition according to any one of embodiments 31 to 34, wherein the at least one viscosity agent is present in an amount of 1% or less by weight of the composition.

Embodiment 36. The composition according to any one of embodiments 31 to 35, wherein the at least one viscosity agent is present in an amount ranging from 0.1% to 0.3% by weight of the composition.

Embodiment 37. The composition according to any one of embodiments 31 to 36, wherein the at least one viscosity agent is present in an amount of 0.15%, 0.16%, 0.17%, 0.18%, 0.19%, 0.2%, 0.21%, 0.22%, 0.23%, 0.24%, or 0.25%.

Embodiment 38. The composition according to any one of embodiments 1 to 37, further comprising at least one penetration enhancer.

Embodiment 39. The composition according to embodiment 38, wherein the at least one penetration enhancer is chosen from benzyl alcohol, diethylene glycol monoethyl ether, caprylic acid, and sodium oleate.

Embodiment 40. The composition according to any one of embodiments 1 to 39, further comprising at least one stabilizing agent.

Embodiment 41. The composition according to embodiment 40, wherein the at least one stabilizing agent is chosen from ascorbic acid and butylated hydroxytoluene.

Embodiment 42. The composition according to any one of embodiments 1 to 41, further comprising at least one solubilizing agent.

Embodiment 43. The composition according to embodiment 42, wherein the at least one solubilizing agent is chosen from tocopherols, fixed oils, soybean oil, PEG-15 hydroxystearates, polysorbate 20, polysorbate 80, 2-hydroxypropyl-β-cyclodextrin, and γ-cyclodextrin.

Embodiment 44. The composition according to embodiment 43, wherein the fixed oils are chosen from corn oil, cottonseed oil, peanut oil, and sesame oil.

Embodiment 45. The composition according to any one of embodiments 1 to 44, further comprising at least one encapsulating agent.

Embodiment 46. The composition according to embodiment 45, wherein the at least one encapsulating agent is chosen from 1,2-dimyristoyl-sn-glycero-phosphocholine, 1,2-distearoyl-sn-glycero-3-(phosphor-rac-(1-glycerol)), 1,2-distearoyl-sn-glycero-3-phosphocholine, cholesterol, DL-dipalmitoylphosphatidylglycerol, sodium N-(carbonyl-methoxypolyethylene glycol 2000)-1,2-distearoyl-sn-glycero-3-phosphoethanolamine, sodium N-(carbonyl-methoxypolyethylene glycol 2000)-distearoyl-glycerophosphoethanolamine, DL-distearoylphosphatidylcholine, egg phospholipids, and hydrogenated soybean lecithin.

Embodiment 47. The composition according to any one of embodiments 1 to 46, wherein the composition has a pH ranging from 5 to 7.

Embodiment 48. The composition according to any one of embodiments 1 to 47, wherein the composition has a pH of 5.2, 5.3, 5.4, 5.5, 5.6, 5.7, 5.8, 5.9, 6, 6.1, or 6.2.

Embodiment 49. The composition according to any one of embodiments 1 to 48, further comprising at least one imaging agent.

Embodiment 50. The composition according to embodiment 49, wherein the at least one imaging agent is a radiographic contrast agent.

Embodiment 51. The composition according to any one of embodiments 1 to 50, further comprising at least one anti-proliferative agent.

Embodiment 52. The composition according to embodiment 51, wherein the at least one anti-proliferative agent is paclitaxel.

Embodiment 53. The composition according to any one of embodiments 1 to 48, wherein the at least one active agent does not degrade 52 weeks after preparation of the composition.

Embodiment 54. The composition according to any one of embodiments 1 to 48 or 53, wherein there is 0% to less than 0.5% by weight of impurities present in the composition 52 weeks after preparation of the composition.

Embodiment 55. The composition according to any one of embodiments 1 to 48 or 53, wherein there is 0% to 0.5% by weight of impurities present in the composition eight weeks after preparation of the composition.

Embodiment 56. The composition according to any one of embodiments 1 to 48 or 53, wherein the total amount of impurities in the composition 156 weeks after preparation of the composition is less than three-fold the total amount of impurities initially present in the composition.

Embodiment 57. The composition according to any one of embodiments 1 to 48 or 53, wherein the total amount of impurities in the composition 52 weeks after preparation of the composition is less than three-fold the total amount of impurities initially present in the composition.

Embodiment 58. The composition according to any one of embodiments 49 to 52, wherein the at least one active agent does not degrade one week after preparation of the composition.

Embodiment 59. The composition according to any one of embodiments 49 to 52 or 58, wherein there is 0% to 0.5% by weight of impurities present in the composition one week after preparation of the composition.

Embodiment 60. The composition according to embodiment 49 to 52 or 58, wherein the total amount of impurities in the composition one week after preparation of the composition is less than three-fold the total amount of impurities initially present in the composition.

Embodiment 61. The composition according to embodiment 49 to 52 or 58, wherein the at least one active agent does not degrade one day after preparation of the composition.

Embodiment 62. The composition according to any one of embodiments 49 to 52, 58, or 61, wherein there is 0% to 0.5% by weight of impurities present in the composition one day after preparation of the composition.

Embodiment 63. The composition according to embodiment 49 to 52, 58, or 61, wherein the total amount of impurities in the composition one day after preparation of the composition is less than three-fold the total amount of impurities initially present in the composition.

Embodiment 64. A composition chosen from one of the compositions recited in Table 1.

Embodiment 65. A composition according to any one of embodiments 1-64, wherein the composition is stored for a period of 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks.

Embodiment 66. A composition according to embodiment 65, wherein the composition is stored at a temperature ranging from 1° C. to 40° C.

Embodiment 67. A composition according to embodiment 66, wherein the composition is stored at 5° C., at 25° C., at 40° C., or at a temperature ranging from 25° C. to 40° C.

Embodiment 68. A composition according to any one of embodiments 65-67, wherein there is 0% to less than 15%, less than 10%, less than 5%, less than 2%, less than 1%, less than 0.5%, less than 0.25%, less than 0.1%, or less than 0.05% by weight of impurities present in the composition after storage.

Embodiment 69. A composition according to any one of embodiments 65-67, wherein the total amount of impurities in the composition is less than five-fold, less than four-fold, less than three-fold, or less than two-fold the total amount of impurities initially present in the composition before storage.

Embodiment 70. A composition according to any one of embodiments 65-67, wherein the at least one active agent of the composition does not degrade during storage.

Embodiment 71. A composition according to any one of embodiments 65-67, wherein the storage does not affect the percentage of impurities present in the composition.

In order that the disclosure herein may be more fully understood, the following examples are set forth. It should be understood that these examples are for illustrative purposes only and are not to be construed as limiting this disclosure in any manner.

### EXAMPLES

### Abbreviations

The following abbreviations are used herein:
HPLC=high pressure liquid chromatography
TFA=trifluoroacetic acid
UPLC=ultra performance liquid chromatography

General methods and experimental details for preparing Compound of Formula (I) of the present disclosure are set forth below.

Reagents

The excipients and reagents used in the following examples are compendial or pharmaceutical grade, or higher.

Purity Assessment

The purity of a composition was analyzed by UPLC on a 2.1×100 mm C18 column with 1.7 μm packing. The analytical method was a 7 minute gradient at 30° C. and a flowrate of 0.61 mL/min consisting of acetonitrile, water, and trifluoracetic acid. Absorption was measured at 210 nm and 440 nm.

### Example 1: Formulations

Table 1 lists non-limiting exemplary formulations.

TABLE 1
Exemplary formulations.

| | Component Concentration (% w/w) | | | | | |
|---|---|---|---|---|---|---|
| | Form. | Form. | Form. | Form. | Form. | Form. |
| Component | #1 | #2 | #3 | #4 | #5 | #6 |
| Diacetate Salt of | 0.115-1.1 | 0.115-1.1 | 0.115-1.1 | 0.115-1.1 | 0.115-1.1 | 0.115-1.1 |
| Compound of Formula (I) | | | | | | |
| Potassium Chloride | 0-0.02 | 0-0.02 | 0-0.02 | 0 | 0-0.02 | 0-0.02 |
| Sodium Chloride | 0.4-0.9 | 0.4-0.9 | 0.4-0.9 | 0.1-0.9 | 0.4-0.9 | 0.4-0.9 |
| Sodium Phosphate Dibasic | 0.05-0.2 | 0.01-0.04 | 0.05-0.2 | 0 | 0.05-0.2 | 0.01-0.04 |
| (anhydrous) | | | | | | |
| Potassium Phosphate | 0.01-0.04 | 0.05-0.2 | 0.01-0.04 | 0 | 0.01-0.04 | 0.05-0.2 |
| Monobasic | | | | | | |
| (anhydrous) | | | | | | |
| Methyl cellulose | 0 | 0 | 0 | 0 | 0-0.5 | 0-0.5 |
| Sodium carboxy methyl | 0 | 0 | 0 | 0 | 0-0.5 | 0-0.5 |
| cellulose | | | | | | |
| Acetic Acid | QID to pH | | | 0 | QID to pH | |
| Sodium Hydroxide | QID to pH | | | 0 | QID to pH | |
| Water (water for injection) | QS to 100% | | | | | |
| Formulation pH | 7-7.5 | 6-6.5 | 8-8.5 | 6-7 | 7-7.5 | 6-6.5 |

| | Component Concentration (% w/w) | | | | |
|---|---|---|---|---|---|
| | Form. | Form. | Form. | Form. | Form. |
| Component | #7 | #8 | #9 | #10 | #11 |
| Diacetate Salt of | 0.115-1.1 | 0.115-1.1 | 0.46 | 0.46 | 0.46 |
| Compound of Formula (I) | | | | | |
| Potassium Chloride | 0-0.02 | 0 | 0 | 0 | 0 |
| Sodium Chloride | 0.4-0.9 | 0.1-0.9 | 0.76 | 0.76 | 0.83 |
| Sodium Phosphate Dibasic | 0.05-0.2 | 0 | 0.0245 | 0.0245 | 0 |
| (anhydrous) | | | | | |
| Potassium Phosphate | 0.01-0.04 | 0 | 0.12 | 0.12 | 0 |
| Monobasic | | | | | |
| (anhydrous) | | | | | |
| Methyl cellulose | 0-0.5 | 0-0.5 | 0 | 0.23 | 0 |
| Sodium carboxy methyl | 0-0.5 | 0-0.5 | 0 | 0 | 0 |
| cellulose | | | | | |
| Acetic Acid | QID to pH | 0 | QID to pH | | |
| Sodium Hydroxide | QID to pH | 0 | QID to pH | | |
| Water (water for injection) | QS to 100% | | | | |
| Formulation pH | 8-8.5 | 6-7 | 6 | 6 | 5.3 |

The non-limiting exemplary formulations listed in Table 1 were prepared by adding the tonicity agent(s) and buffer components to water (water for injection grade) at the desired concentration. If present, the viscosity agent(s) is added with buffer components. The resulting mixture was then mixed at room temperature until a colorless, clear mixture was obtained. Diacetate salt of Compound of Formula (I) was then added in the desired amount and the resulting mixture was mixed at room temperature until a yellow/orange translucent mixture was obtained. An aliquot of the mixture was then obtained and the pH was measured using a pH electrode. If the pH was found to be outside of the desired pH range (as indicated in Table 1), the pH was adjusted by addition of acid or base. The resulting mixture is filtered through a 0.2 micron sterile filter system and stored in a sterile vial.

### Example 2: Stability Studies

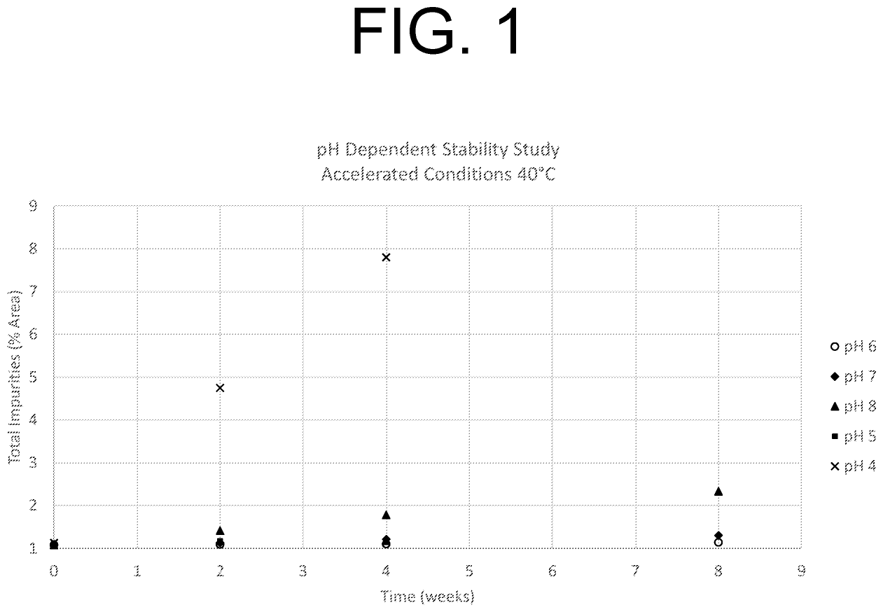

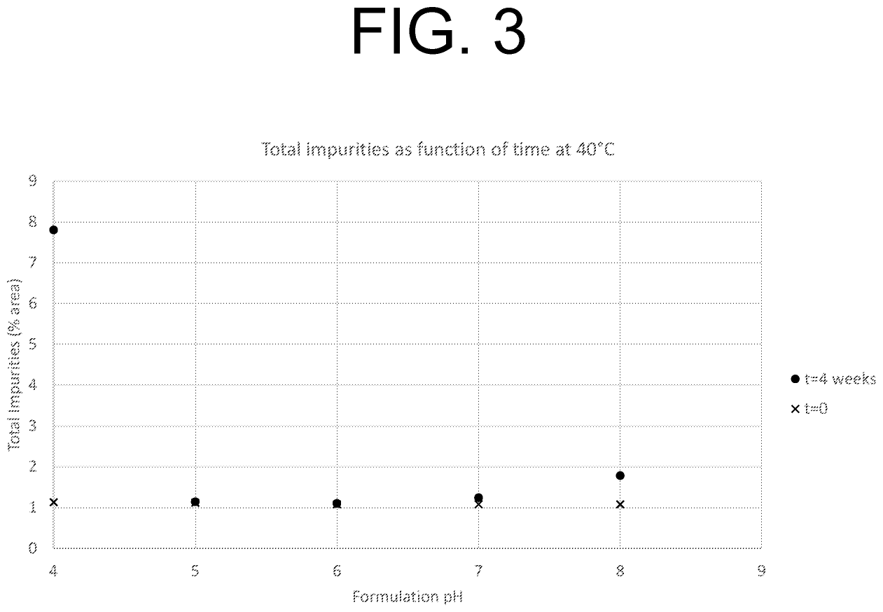

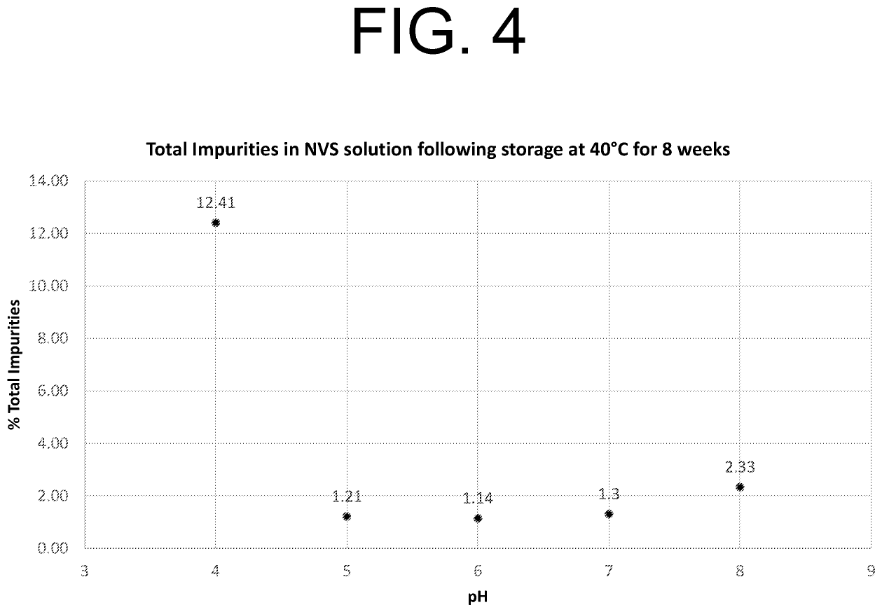

To determine the stability of the formulations described herein, the amount of impurities was measured. A formulation was added to a 10 mL amber vial and stored at 40° C. in a constant temperature chamber. Parameters such as length of storage and formulation pH were varied, as shown in FIGS. 1-4. At the desired time point(s), the formulation was analyzed by UPLC and the pH was measured. No change in physical appearance was observed.

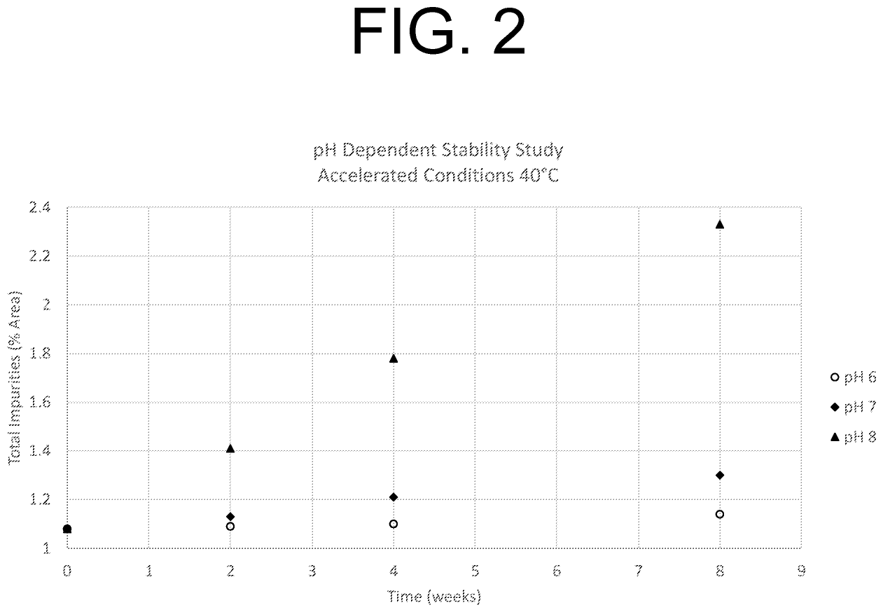

As shown in FIGS. 1-4, the formulations with pH 4 or pH 8 exhibited the greatest concentration of impurities at any given time point. It was surprisingly found that formulations having a pH of 6 resulted in the least amount of impurities. See, e.g., FIGS. 2-4.

Additional studies were performed to test the stability of a formulation of compound I, prepared at 4 mg/mL, and stored at three different temperatures for 6-12 months (Tables 2-4). Table 2 summarizes the results of a study where the formulation was stored at 5±3° C. for 12 months. Table 3 summarizes the results of a study where the formulation was stored at 25±2° C./60±5% RH for 12 months. And Table 4 summarizes the results of a study where the formulation was stored at 40±2° C./75±5 RH for 6 months. During storage, samples were taken at the desired time points and analyzed. No change in physical appearance was observed. In all three studies the pH of the formulation remained constant throughout the 6 or 12 months of storage. The concentration of impurities in the formulation was also measured at each time point, showing no change in the chemical and physical parameters measured throughout the 6 or 12 months of storage.

TABLE 2
Stability data for compound I formulation stored at 5 ± 3° C.

| | Target | Time Points (Month) | | | | | |
|---|---|---|---|---|---|---|---|
| Test | Criteria | Initial | 1 | 3 | 6 | 9 | 12 |
| Appearance | Clear, | Clear, | Clear, | Clear, | Clear, | Clear, | Clear, |
| Observation | orange | orange | orange | orange | orange | orange | orange |
| | solution, | solution, | solution, | solution, | solution, | solution, | solution, |
| | free from | free from | free from | free from | free from | free from | free from |
| | visible | visible | visible | visible | visible | visible | visible |
| | particulates | particulates | particulates | particulates | particulates | particulates | particulates |
| pH | Report | 6.0 | 6.0 | 6.0 | 6.0 | 6.0 | 6.0 |
| Assay. ALU- | 85-115% | 93% | 102% | 98% | 99% | 103% | 102% |
| TM-001 | | | | | | | |
| Purity. ALU- | >95% | 98.9% | 98.9% | 98.9% | 98.9% | 98.9% | 99.0% |
| TM-001 | | | | | | | |
| Impurities: | Record: | 1.04/1.05% | 1.04/1.05% | 1.04/1.07% | 1.04/1.06% | 1.04/1.07% | 1.04/1.04% |
| individual | RRT, | 1.02/0.05% | 1.02/0.08% | 1.02/0.07% | 1.02/0.07% | 1.02/0.08% | |
| related. | Area % | | | | | | |
| ALU-TM- | | | | | | | |
| 001 | | | | | | | |
| Osmolality | 270-300 | 282 | 282 | 284 | 289 | 287 | 288 |
| | mOsm/kg | | | | | | |
| Particulate | Particle | Particles ≥ | | Particles ≥ | Particles ≥ | | Particles ≥ |
| Analysis*** | size ≥ 10 | 10 μm: | | 10 μm: | 10 μm: | | 10 μm: |
| Light | μm: ≤ 6,000 | 240/Vial | | 272/Vial | 158/Vial | | 220/Vial |
| Obscuration | Particles/ | 154/Vial | | 482/Vial | 96/Vial | | 192/Vial |
| Particle | Vial | Particles ≥ | | Particles ≥ | Particles ≥ | | Particles ≥ |
| Count | Particle | 25 μm: | | 25 μm: | 25 μm: | | 25 μm: |
| | size ≥ | 14/Vial | | 25/Vial | 2/Vial | | 4/Vial |
| | 25 μm: ≤ 600 | 4/Vial | | 14/Vial | 0/Vial | | 0/Vial |
| | Particles/ | | | | | | |
| | Vial | | | | | | |

***Modified USP 788 to capture counts in individual vials.

TABLE 3
Stability data for compound I formulation stored at 25 ± 2° C./60 ± 5% RH

| | Target | Time Points (Month) | | | | | |
|---|---|---|---|---|---|---|---|
| Test | Criteria | Initial | 1 | 3 | 6 | 9 | 12 |
| Appearance | Clear, | Clear, | Clear, | Clear, | Clear, | Clear, | Clear, |
| Observation | orange | orange | orange | orange | orange | orange | orange |
| | solution, | solution, | solution, | solution, | solution, | solution, | solution, |
| | free from | free from | free from | free from | free from | free from | free from |
| | visible | visible | visible | visible | visible | visible | visible |
| | particulates | particulates | particulates | particulates | particulates | particulates | particulates |
| pH | Report | 6.0 | 6.0 | 6.0 | 6.1 | 6.0 | 6.1 |
| Assay. ALU- | 85-115% | 93% | 103% | 99% | 98% | 104% | 100% |
| TM-001 | | | | | | | |
| Purity. ALU- | >95% | 98.9% | 98.9% | 98.9% | 98.8% | 98.9% | 98.9% |
| TM-001 | | | | | | | |
| Impurities: | Record: | 1.04/1.05% | 1.04/1.06% | 1.04/1.08% | 1.04/1.10% | 1.04/1.08% | 1.04/1.06% |
| individual | RT (min), | 1.02/0.05% | 1.02/0.08% | 1.02/0.07% | 1.02/0.06% | 1.02/0.07% | |
| related. | Area% | | | | | | |
| ALU- TM- | | | | | | | |
| 001 | | | | | | | |
| Osmolality | 270 - 300 | 282 | 284 | 281 | 289 | 285 | 288 |
| | mOsm/kg | | | | | | |
| Particulate | Particle | Particles ≥ | NT | Particles ≥ | Particles ≥ | NT | Particles ≥ |
| Analysis*** | size ≥ 10 | 10 μm: | | 10 μm: | 10 μm: | | 10 μm: |
| Light | μm: ≤ 6,000 | 240/Vial | | 570/Vial | 272/Vial | | 434/Vial |
| Obscuration | Particles/ | 154/Vial | | 694/Vial | 548/Vial | | 490/Vial |
| Particle | Vial | Particles ≥ | | Particles ≥ | Particles ≥ | | Particles ≥ |
| Count | Particle | 25 μm: | | 25 μm: | 25 μm: | | 25 μm: |
| | size ≥ | 14/Vial | | 22/Vial | 4/Vial | | 4/Vial |
| | 25 μm: ≤ 600 | 4/Vial | | 20/Vial | 8/Vial | | 8/Vial |
| | Particles/ | | | | | | |
| | Vial | | | | | | |

***Modified USP 788 to capture counts in individual vials.

TABLE 4
Stability data for compound 1 formulation stored at 40 ± 2° C./75 ± 5 RH

| | | Time Points (Month) | | | |
|---|---|---|---|---|---|
| Test | Target Criteria | Initial | 1 Month | 3 Months | 6 Months |
| Appearance | Clear, orange | Clear, orange | Clear, orange | Clear, orange | Clear, orange |
| Observation | solution, | solution, | solution, | solution, | solution, |
| | free from | free from | free from | free from | free from |
| | visible | visible | visible | visible | visible |
| | particulates | particulates | particulates | particulates | particulates |
| pH | Report | 6.0 | 6.0 | 6.0 | 6.1 |
| Assay. ALU- | 85-115% | 93% | 104% | 90% | 100% |
| TM-001 | | | | | |
| Purity. ALU- | >95% | 98.9% | 98.9% | 98.8% | 98.8% |
| TM-001 | | | | | |
| Impurities: | Record: RT (min), | 1.04/1.05% | 1.04/1.07% | 1.04/1.14% | 1.04/1.11% |
| individual | Area % | 1.02/0.05% | 1.02/0.06% | 1.02/0.08% | 1.02/0.06% |
| related. | | | | | |
| ALU- TM- | | | | | |
| 001 | | | | | |
| Osmolality | 270-300 | 282 | 285 | 280 | 288 |
| | mOsm/kg | | | | |

### Example 3: Combination of an Exemplary Composition and a Radiographic Contrast Agent

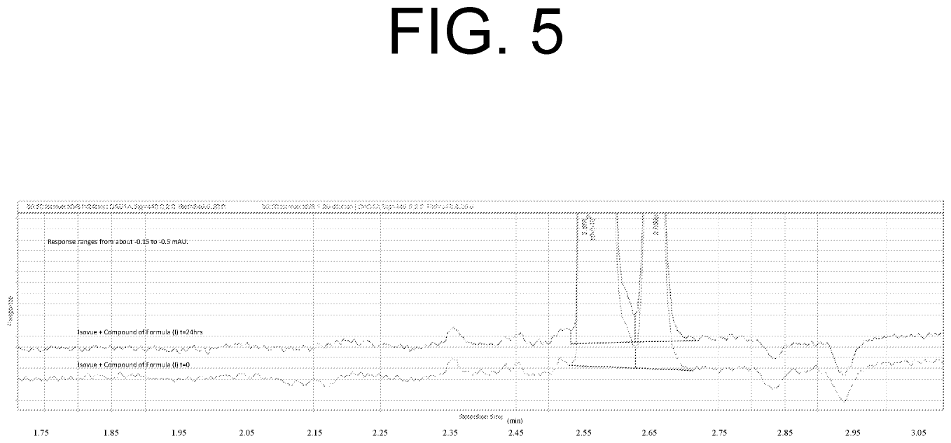

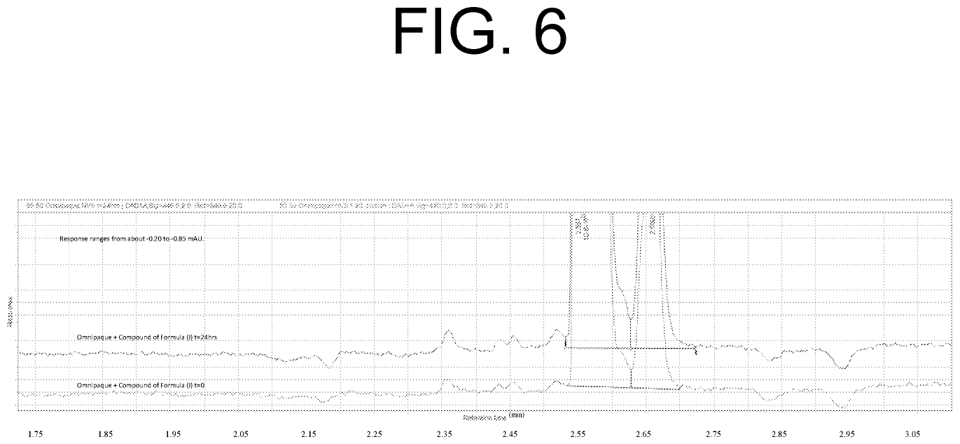

Equal volumes of a formulation from Example 1 and radiographic contrast agent (Omnipaque or Isovue), as received from the manufacturer, were drawn into separate syringes. The two syringes were connected to an indeflator by a three-way joint/stopcock. The indeflator was evacuated and the mixtures from both syringes were transferred into the indeflator. The indeflator was then inverted six times to ensure complete mixing of the mixtures. The resulting mixture was a yellow/orange translucent mixture, stored in an amber scintillation vial at room temperature, and analyzed by UPLC upon storage and after 24 hours. The results are summarized in Table 5 and shown in FIGS. 5 and 6. It was surprisingly found that the Compound of Formula (I) is stable after 24 hours when combined with a radiographic contrast agent.

TABLE 5
UPLC analytical results of a combination of an exemplary
composition and a radiographic contrast agent.

| | t = 0 hours | | t = 24 hours | |
|---|---|---|---|---|
| Radio- | Compound of | Total | Compound of | Total |
| graphic | Formula (I) | impurities | Formula (I) | impurities |
| Agent | (HPLC | (HPLC | (HPLC | (HPLC |
| Contrast | area-%) | area-%) | area-%) | area-%) |
| Isovue | 98.98 | 1.02 | 98.97 | 1.03 |
| Omnipaque | 98.98 | 1.02 | 98.95 | 1.05 |

### Example 4: Combination of an Exemplary Composition and an Anti-Proliferative Agent

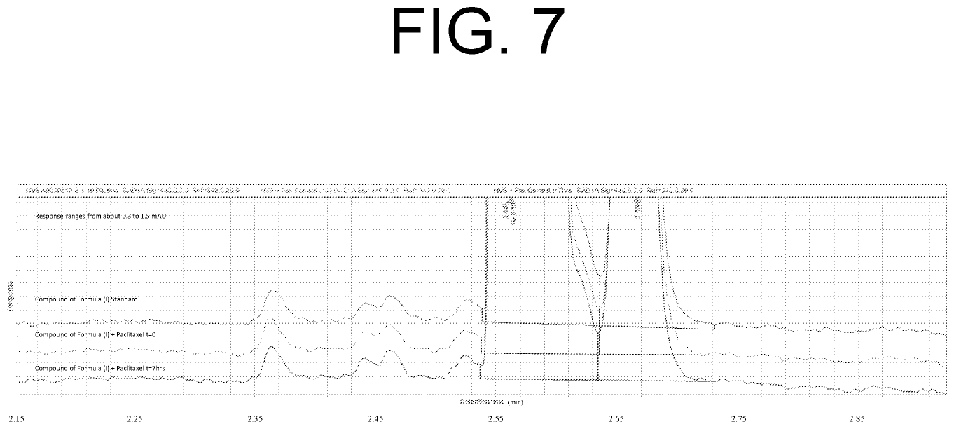

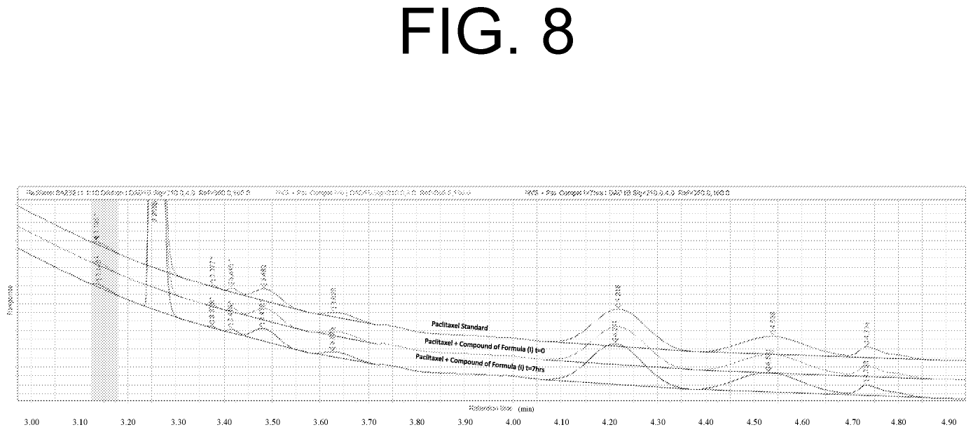

Equal volumes of a formulation from Example 1 and paclitaxel injection solution (6 mg/mL; used as received from manufacturer) were combined in an amber scintillation vial and stored at room temperature. The resulting mixture was a clear yellow solution. An aliquot was obtained, diluted 1:10 with 0.1% aqueous TFA and analyzed by UPLC as described herein. Another aliquot was obtained, 7 hours after preparation of the mixture, and similarly diluted and analyzed. The results are summarized in Table 6 and shown in FIGS. 7 and 8. It was surprisingly found that the Compound of Formula (I) and paclitaxel are stable seven hours after being combined in the same composition.

TABLE 6
UPLC analytical results of a combination
of an exemplary composition and paclitaxel.

| | Compound of | Total |
|---|---|---|
| | Formula (I) | impurities |
| Time | (HPLC area-%) | (HPLC area-%) |
| 0 hours | 98.9 | 1.1 |
| 1 hour | 98.9 | 1.1 |
| 4 hours | 98.9 | 1.1 |
| 7 hours | 98.9 | 1.1 |

What is claimed is:

1. A composition comprising:
at least one active agent chosen from a Compound of Formula (I): 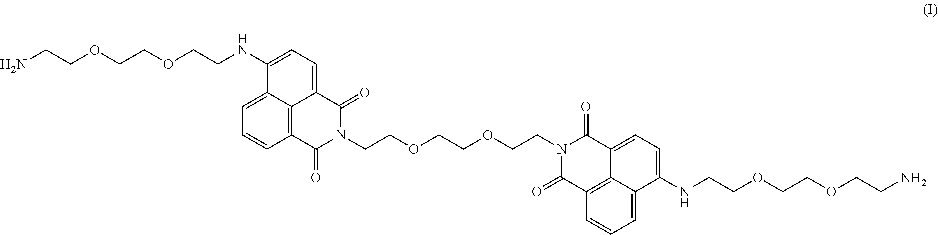 [SMILES: NCCOCCOCCNc1ccc2c3c(cccc13)C(=O)N(CCOCCOCCN1C(=O)c3cccc4c(NCCOCCOCCN)ccc(c34)C1=O)C2=O]
and pharmaceutically acceptable salts thereof;
at least one tonicity agent;
at least one buffer; and
at least one vehicle.

2. The composition according to claim 1, wherein the at least one active agent is 2,2′-((((((((ethane-1,2-diylbis(oxy))bis(ethane-2,1-diyl))bis(1,3-dioxo-2,3-dihydro-1H-benzo[de]isoquinoline-2,6-diyl))bis(azanediyl))bis(ethane-2,1-diyl))bis(oxy))bis(ethane-2,1-diyl))bis(oxy))bis(ethan-1-aminium) diacetate.

3. The composition according to claim 1 or 2, wherein the at least one active agent is present in an amount ranging from 0.01% to 2.5% by weight of the composition.

4. The composition according to any one of claims 1 to 3, wherein the at least one active agent is present in an amount of 0.01%, 0.02%, 0.03%, 0.04%, 0.05%, 0.06%, 0.07%, 0.08%, 0.09%, 0.1%, 0.12%, 0.14%, 0.16%, 0.18%, 0.2%, 0.22%, 0.24%, 0.26%, 0.28%, 0.3%, 0.32%, 0.34%, 0.36%, 0.38%, 0.4%, 0.42%, 0.44%, 0.46%, 0.48%, 0.5%, 0.52%, 0.54%, or 0.56% by weight of the composition.

5. The composition according to any one of claims 1 to 4, wherein the at least one active agent is present in an amount of 0.16%, 0.18%, 0.2%, 0.22%, 0.24%, 0.26%, 0.28%, 0.3%, 0.32%, 0.34%, 0.36%, 0.38%, 0.4%, 0.42%, 0.44%, 0.46%, 0.48%, or 0.5% by weight of the composition.

6. The composition according to any one of claims 1 to 5, wherein the at least one tonicity agent is chosen from dextrose, sorbitol, lactose, mannitol, sodium chloride, potassium chloride, and glycerol.

7. The composition according to any one of claims 1 to 6, wherein the at least one tonicity agent is sodium chloride.

8. The composition according to any one of claims 1 to 6, wherein the at least one tonicity agent is potassium chloride.

9. The composition according to any one of claims 1 to 8, wherein the at least one tonicity agent is present in an amount up to 2% by weight of the composition.

10. The composition according to any one of claims 1 to 9, wherein the at least one tonicity agent is present in an amount of 0.5% to 1.5% by weight of the composition.

11. The composition according to any one of claims 1 to 10, wherein the at least one tonicity agent is present in an amount of 0.6%, 0.61%, 0.62%, 0.63%, 0.64%, 0.65%, 0.66%, 0.67%, 0.68%, 0.69%, 0.7%, 0.71%, 0.72%, 0.73%, 0.74%, 0.75%, 0.76%, 0.77%, 0.78%, 0.79%, 0.8%, 0.81%, 0.82%, 0.83%, 0.84%, 0.85%, 0.86%, 0.87%, 0.88%, 0.89%, or 0.9% by weight of the composition.

12. The composition according to any one of claims 1 to 11, wherein the at least one tonicity agent is present in an amount of 0.72%, 0.74%, 0.76%, 0.78%, 0.8%, 0.82%, or 0.84% by weight of the composition.

13. The composition according to any one of claims 1 to 12, wherein the at least one buffer is chosen from a potassium salt, a sodium salt, and maleic acid.

14. The composition according to claim 13, wherein the potassium salt is chosen from potassium phosphate monobasic and potassium phosphate dibasic.

15. The composition according to claim 13, wherein the sodium salt is chosen from sodium phosphate monobasic and sodium phosphate dibasic.

16. The composition according to any one of claims 1 to 15, wherein the at least one buffer is present in an amount up to 1% by weight of the composition.

17. The composition according to any one of claims 1 to 16, wherein the at least one buffer is present in an amount ranging from 0.05% to 0.4% by weight of the composition.

18. The composition according to any one of claims 1 to 17, wherein the at least one buffer is present in an amount of 0.06%, 0.08%, 0.1%, 0.12%, 0.14%, 0.16%, 0.18%, 0.2%, 0.22%, 0.24%, 0.26%, 0.28%, or 0.3% by weight of the composition.

19. The composition according to any one of claims 1 to 17, wherein the at least one buffer is present in an amount of 0.08% to 0.16% by weight of the composition.

20. The composition according to any one of claims 1 to 17, wherein the at least one buffer comprises a sodium salt in an amount ranging from 0.01% to 0.03% by weight of the composition and a potassium salt in an amount ranging from 0.1% to 0.14% by weight of the composition.

21. The composition according to claim 20, wherein the sodium salt is sodium phosphate dibasic.

22. The composition according to claim 20, wherein the potassium salt is potassium phosphate monobasic.

23. The composition according to any one of claims 1 to 22, wherein the at least one vehicle is chosen from water, ethanol, isopropanol, polyethylene glycols, and propylene glycols.

24. The composition according to claim 23, wherein the polyethylene glycols are chosen from polyethylene glycol 300, polyethylene glycol 400, and polyethylene glycol 600.

25. The composition according to any one of claims 1 to 24, wherein the at least one vehicle is water.

26. The composition according to any one of claims 1 to 25, further comprising at least one pH modulation agent.

27. The composition according to claim 26, wherein the at least one pH modulation agent is chosen from acetic acid, carbonic acid, citric acid, sodium bicarbonate, and sodium hydroxide.

28. The composition according to claim 26 or 27, wherein the at least one pH modulation agent comprises acetic acid.

29. The composition according to any one of claims 26 to 28, wherein the at least one pH modulation agent comprises sodium hydroxide.

30. The composition according to any one of claims 1 to 29, further comprising at least one viscosity agent.

31. The composition according to claim 30, wherein the at least one viscosity agent is chosen from gelatin, methylcellulose, hydroxypropyl cellulose, hydroxypropyl methylcellulose, hydroxyethyl cellulose, hydroxypropylmethyl cellulose, methylhydroxyethyl cellulose, methylhydroxypropyl cellulose, hydroxyethylcarboxymethyl cellulose, carboxymethyl cellulose, carboxymethylhydroxyethyl cellulose, and sodium carboxymethylcellulose.

32. The composition according to claim 31, wherein the at least one viscosity agent is methyl cellulose.

33. The composition according to claim 31, wherein the at least one viscosity agent is sodium carboxymethyl cellulose.

34. The composition according to claim 31, wherein the at least one viscosity agent is hydroxypropyl cellulose.

35. The composition according to any one of claims 31 to 34, wherein the at least one viscosity agent is present in an amount of 1% or less by weight of the composition.

36. The composition according to any one of claims 31 to 35, wherein the at least one viscosity agent is present in an amount ranging from 0.1% to 0.3% by weight of the composition.

37. The composition according to any one of claims 31 to 36, wherein the at least one viscosity agent is present in an amount of 0.15%, 0.16%, 0.17%, 0.18%, 0.19%, 0.2%, 0.21%, 0.22%, 0.23%, 0.24%, or 0.25%.

38. The composition according to any one of claims 1 to 37, further comprising at least one penetration enhancer.

39. The composition according to claim 38, wherein the at least one penetration enhancer is chosen from benzyl alcohol, diethylene glycol monoethyl ether, caprylic acid, and sodium oleate.

40. The composition according to any one of claims 1 to 39, further comprising at least one stabilizing agent.

41. The composition according to claim 40, wherein the at least one stabilizing agent is chosen from ascorbic acid and butylated hydroxytoluene.

42. The composition according to any one of claims 1 to 41, further comprising at least one solubilizing agent.

43. The composition according to claim 42, wherein the at least one solubilizing agent is chosen from tocopherols, fixed oils, soybean oil, PEG-15 hydroxystearates, polysorbate 20, polysorbate 80, 2-hydroxypropyl-β-cyclodextrin, and γ-cyclodextrin.

44. The composition according to claim 43, wherein the fixed oils are chosen from corn oil, cottonseed oil, peanut oil, and sesame oil.

45. The composition according to any one of claims 1 to 44, further comprising at least one encapsulating agent.

46. The composition according to claim 45, wherein the at least one encapsulating agent is chosen from 1,2-dimyristoyl-sn-glycero-phosphocholine, 1,2-distearoyl-sn-glycero-3-(phosphor-rac-(1-glycerol)), 1,2-distearoyl-sn-glycero-3-phosphocholine, cholesterol, DL-dipalmitoylphosphatidylglycerol, sodium N-(carbonyl-methoxypolyethylene glycol 2000)-1,2-distearoyl-sn-glycero-3-phosphoethanolamine, sodium N-(carbonyl-methoxypolyethylene glycol 2000)-distearoyl-glycerophosphoethanolamine, DL-distearoylphosphatidylcholine, egg phospholipids, and hydrogenated soybean lecithin.

47. The composition according to any one of claims 1 to 46, wherein the composition has a pH ranging from 5 to 7.

48. The composition according to any one of claims 1 to 47, wherein the composition has a pH of 5.2, 5.3, 5.4, 5.5, 5.6, 5.7, 5.8, 5.9, 6, 6.1, or 6.2.

49. The composition according to any one of claims 1 to 48, further comprising at least one imaging agent.

50. The composition according to claim 49, wherein the at least one imaging agent is a radiographic contrast agent.

51. The composition according to any one of claims 1 to 50, further comprising at least one anti-proliferative agent.

52. The composition according to claim 51, wherein the at least one anti-proliferative agent is paclitaxel.

53. The composition according to any one of claims 1 to 48, wherein the at least one active agent does not degrade 52 weeks after preparation of the composition.

54. The composition according to any one of claim 1 to 48 or 53, wherein there is 0% to less than 0.5% by weight of impurities present in the composition 52 weeks after preparation of the composition.

55. The composition according to any one of claim 1 to 48 or 53, wherein there is 0% to 0.5% by weight of impurities present in the composition eight weeks after preparation of the composition.

56. The composition according to any one of claim 1 to 48 or 53, wherein the total amount of impurities in the composition 156 weeks after preparation of the composition is less than three-fold the total amount of impurities initially present in the composition.

57. The composition according to any one of claim 1 to 48 or 53, wherein the total amount of impurities in the composition 52 weeks after preparation of the composition is less than three-fold the total amount of impurities initially present in the composition.

58. The composition according to any one of claims 49 to 52, wherein the at least one active agent does not degrade one week after preparation of the composition.

59. The composition according to any one of claim 49 to 52 or 58, wherein there is 0% to 0.5% by weight of impurities present in the composition one week after preparation of the composition.

60. The composition according to claim 49 to 52 or 58, wherein the total amount of impurities in the composition one week after preparation of the composition is less than three-fold the total amount of impurities initially present in the composition.

61. The composition according to claim 49 to 52 or 58, wherein the at least one active agent does not degrade one day after preparation of the composition.

62. The composition according to any one of claim 49 to 52, 58, or 61, wherein there is 0% to 0.5% by weight of impurities present in the composition one day after preparation of the composition.

63. The composition according to claim 49 to 52, 58, or 61, wherein the total amount of impurities in the composition one day after preparation of the composition is less than three-fold the total amount of impurities initially present in the composition.

64. A composition chosen from one of the compositions recited in Table 1.

65. A composition according to any one of claims 1-64, wherein the composition is stored for a period of 4 weeks, 5 weeks, 6 weeks, 7 weeks, 8 weeks, 9 weeks, 10 weeks, 11 weeks, 12 weeks, 16 weeks, 20 weeks, 24 weeks, 28 weeks, 32 weeks, 36 weeks, 40 weeks, 44 weeks, 48 weeks, 52 weeks, 78 weeks, 104 weeks, 130 weeks, 156 weeks, 182 weeks, or 208 weeks.

66. A composition according to claim 65, wherein the composition is stored at a temperature ranging from 1° C. to 40° C.

67. A composition according to claim 66, wherein composition is stored at 5° C., at 25° C., at 40° C., or at a temperature ranging from 25° C. to 40° C.

68. A composition according to any one of claims 65-67, wherein 0% to less than 15%, to less than 10%, to less than 5%, to less than 2%, to less than 1%, to less than 0.5%, to less than 0.25%, to less than 0.1%, or to less than 0.05% by weight of impurities are present in the composition after storage.

69. A composition according to any one of claims 65-67, wherein the total amount of impurities in the composition is less than five-fold, less than four-fold, less than three-fold, or less than two-fold the total amount of impurities initially present in the composition before storage.

70. A composition according to any one of claims 65-67, wherein the at least one active agent of the composition does not degrade during storage.

71. A composition according to any one of claims 65-67, wherein the storage does not affect the percentage of impurities present in the composition.
